(19) United States
(12) Patent Application Publication
(10) Pub. No.: US 2020/0172549 A1
(43) Pub. Date: Jun. 4, 2020

(54) NOVEL CHLORIN E6-CURCUMIN DERIVATIVES, PREPARATION METHOD THEREOF, AND PHARMACEUTICAL COMPOSITION CONTAINING THE SAME FOR TREATMENT OF CANCER

(71) Applicant: DONG SUNG PHARM. CO., LTD., Seoul (KR)

(72) Inventors: Shivakumar S JALDE, Daegu (KR); Yong Wan KIM, Seoul (KR); Kwang Hee SON, Gyeongsangbuk-do (KR); Hwan Suk LEE, Gwangju (KR)

(73) Assignee: DONG SUNG PHARM. CO., LTD.

(21) Appl. No.: 16/724,703

(22) Filed: Dec. 23, 2019

Related U.S. Application Data
(63) Continuation of application No. PCT/KR2018/007124, filed on Jun. 22, 2018.

(30) Foreign Application Priority Data
Jun. 23, 2017 (KR) 10-2017-0080023

(51) Int. Cl. C07D 487/22

(52) U.S. Cl. CPC C07D 487/22

(57) ABSTRACT

The present disclosure relates to novel chlorine e6-curcumin derivatives, a preparation method thereof for the treatment of cancer, and in particularly, novel compounds were prepared by using different linkers such as hydrophobic and hydrophilic linkers to conjugate chlorine e6 to curcumin, the compounds under investigation showed excellent photophysical properties, stability, and anticancer activity.

### CROSS REFERENCE TO RELATED APPLICATIONS

This application is a continuation of PCT Application No. PCT/KR2018/007124, filed on 22 Jun. 2018, which claims the benefit of the Korean Patent Application No. 10-2017-0080023, filed on 23 Jun. 2017. The entire disclosures of the applications identified in this paragraph are incorporated herein by reference.

### BACKGROUND

Photodynamic therapy (PDT) is an attractive modality for the treatment of cancer and other diseases. In the photodynamic reaction, the photosensitizer, or photosensitizing agent, promoted to the excited singlet state using light, decays to the triplet state and generates highly reactive oxygen species (ROS) such as singlet oxygen through intermolecular triplet-triplet energy transfer to oxygen (BMCL 2008, 18, 1, 293-297). With no spin-state restriction, singlet oxygen is cytotoxic, readily reacting with electron-rich biomolecules such as unsaturated lipids, amino acids, and DNA, consequently destroying the tumor cell. Antitumor effects of PDT derive from three interrelated mechanisms direct cytotoxic effects on tumor cells, damage to the tumor vasculature and induction of a robust inflammatory reaction that can lead to the development of systemic immunity (CA: A Cancer Journal for Clinicians 2011, 61, 4, 25-281). The molecular mechanisms underlying programmed necrosis are still elusive, but certain events including activation of RIP1 (receptor interacting protein 1) kinase, excessive mitochondrial ROS production, lysosomal damage and intracellular Ca²⁺-overload, are recurrently involved. Severe inner mitochondria membrane photodamage or intracellular Ca²⁺-overload could promote mitochondrial permeability transition, an event that may favor necrotic rather than apoptotic phototoxicity.

PDT has several advantages over other conventional cancer treatments. It is relatively non-invasive because irradiation is limited to the tumor site, and it shows lower systemic toxicity and relatively selective destruction of tumors, partly due to preferential localization of photosensitizer within the tumor. Thus, PDT has been widely employed against various tumors to which irradiation can be applied directly, such as lung, esophageal, gastric, breast, head, and neck, bladder and prostate carcinomas (Anticancer Research 2011, 31, 3, 763-769). When compared with other therapies, PDT often produces the higher cure and lower recurrence rates.

Most of the photosensitizers used in cancer therapy are based on a tetrapyrrole structure, similar to that of the protoporphyrin contained in hemoglobin. An ideal photosensitizing agent should be a single pure compound to allow quality control analysis with low manufacturing costs and good stability in storage. It should have a high absorption peak between 600 and 800-nm (red to deep red) as absorption of photons with wavelengths longer than 800-nm does not provide enough energy to excite oxygen to its singlet state and the capacity for forming a substantial yield of reactive oxygen species upon irradiation. Since the penetration of light into tissue increases with its wavelength, agents with strong absorbance in the deep red such as chlorins, bacteriochlorins, and phthalocyanines offer improvement in tumor control. It should have no dark toxicity and relatively rapid clearance from normal tissues, thereby minimizing phototoxic side-effects.

In our previous embodiment, WO 2013051778 A1 describes the tumor-selective conjugates for the targeted therapy; in particular, chlorine e6 photosensitizer was conjugated with curcumin and folic acid. The conjugates used different linkers to conjugate a targeting agent folic acid, photosensitizer chlorine e6 and natural compound curcumin.

The Indian spice curcumin (also known as diferuloylmethane), extracted from the turmeric plant, has long held a role in Indian/Hindu rituals, traditions, customs, and cuisines (World Journal of Clinical Oncology 2016, 7, 3, 275-283). Some fractions of turmeric, collectively known as curcuminoids (curcumin, demethoxycurcumin, and bisdemethoxycurcumin) are considered to be the active compounds. Curcumin or diferuloylmethane, having molecular weight 368.38, is primary active polyphenolic compounds studied in a host of areas. It is an orange-yellow, crystalline powder and insoluble in water; however, it is highly soluble in ethanol and DMSO. In contrast with conventional cytotoxic drugs which often have side effects such as nausea, vomiting or fatigue curcumin has minimal toxicity. This is a great advantage when treating patients with pancreatic cancer, who generally show poor tolerance to intensive therapy due to their poor clinical conditions. Safety is another advantage of this agent. The safety of curcumin has been approved by the Food and Drug Administration and World Health Organization; In addition, its safety is strongly supported by the fact that this agent has been used in traditional Hindu and Chinese medicine for thousands of years (Cell Division 2015, 10, 6).

Numerous studies have suggested the presence of different metabolites of curcumin. It has been shown to be bio-transformed to dihydrocurcumin and tetrahydrocurcumin. Subsequently, these products are converted to monoglucuronide conjugates. In another study, it was reported that the main biliary metabolites of curcumin are glucuronide conjugates of tetrahydrocurcumin (THC) and hexahydrocurcumin. The other salient feature of turmeric/curcumin is that despite being consumed daily for centuries in Asian countries, it has not been shown to cause any toxicity. Curcumin has been shown to possess a wide range of pharmacological activities including anti-inflammatory, anti-cancer, anti-oxidant, wound healing and anti-microbial effects. Curcumin can modulate the activity of a variety of molecules that play important roles in cancer progression, with more than 30 molecular targets identified to date. Of these molecules, NF-κB appears to be one of the primary targets of curcumin. Curcumin may induce apoptosis of cancer cells through blocking of NF-κB survival pathway, generation of reactive oxygen species (ROS), downregulation of Bcl-XL, or activation of caspase-8 pathways. Recent evidence indicates that curcumin initiates apoptosis through inducing growth arrest and the DNA damage-inducible gene 153 (GADD153), implying curcumin causes DNA damage through topoisomerase H inhibition (Int. J. Onc. 2012, 41, 2184-2190).

Combination therapy in its simplest definition means the use of different modalities that act via different mechanisms in order to produce additive value and, in many cases, a synergistic effect. For example, a combined therapy might work through acting on different cell signaling pathways, enhancing tumor killing efficiency and at the same time blocking cellular resistance capabilities. An inevitable effect of this is the opportunity to reduce the dose of any/all modalities in the therapeutic combination, making it possible to reduce noxious side effects. Conventional cancer therapies, including PDT and chemotherapy as a single modality, have a limited but important role in the overall treatment of most solid tumors. Therefore, the strategies of cancer treatment using combined therapies are considered more promising for higher efficacy, resulting in better survival rates.

### REFERENCE CITED

1. Yao J et al., Design, synthesis, and in vitro photodynamic activities of benzochloroporphyrin derivatives as tumor photosensitizers. Bioorganic medicinal chemistry letters 2008, 18, 1 293-297.
2. Agostinis P et al., Photodynamic therapy of cancer: An update CA: A Cancer Journal for Clinicians 2011, 61, 4, 25-281.
3. Tannaka M et al., Anticancer Effects of Novel Photodynamic Therapy with Glycoconjugated Chlorin for Gastric and Colon Cancer. Anticancer Research 2011, 31, 3, 763-769.
4. Verrna V, Relationship and interactions of curcumin with radiation therapy. World Journal of Clinical Oncology 2016, 7, 3, 275-283.
5. Bose S et al., Curcumin and tumor immune-editing: resurrecting the immune system. Cell Division 2015, 10, 6.
6. Ahn J-C et al., Combination treatment with photodynamic therapy and curcumin induces mitochondria-dependent apoptosis in AMC-HN3 cells Int. J. Onc. 2012, 41, 2184-2190.
7. WO 2013051778 A1: Tumor-selective conjugates for target therapy.

### BRIEF DESCRIPTION OF THE DRAWINGS

FIG. 1 shows a mass spectrum of curcumin butanoic acid (2);

FIG. 2 shows a mass spectrum of dimethylester Ce6 (4);

FIG. 3 shows a mass spectrum of Ce6-Propnane-NHBoc conjugate (5);

FIG. 4 shows a mass spectrum of Ce6-Propnane-amine conjugate (6);

FIG. 5 shows a mass spectrum of chlorine e6-curcumin conjugate (7);

FIG. 6 shows a mass spectrum of Ce6-Hexane-NHBoc conjugate (8);

FIG. 7 shows a mass spectrum of Ce6-Hexane-amine conjugate (9);

FIG. 8 shows a mass spectrum of chlorine e6-curcumin conjugate (10);

FIG. 9 shows a mass spectrum of Ce6-monoPEG-NHBoc conjugate (11);

FIG. 10 shows a mass spectrum of Ce6-monoPEG-amine conjugate (12);

FIG. 11 shows a mass spectrum of chlorine e6-curcumin conjugate (13);

FIG. 12 shows a mass spectrum of Ce6-diPEG-NHBoc conjugate (14);

FIG. 13 shows a mass spectrum of Ce6-diPEG-amine conjugate (15);

FIG. 14 shows a mass spectrum of chlorine e6-curcumin conjugate (16);

FIG. 15 shows dark cytotoxicity data against AsPC-1

FIG. 16 shows dark cytotoxicity data against MIA-PaCa-2

FIG. 17 shows photocytotoxicity data against AsPC-1;

FIG. 18 shows photocytotoxicity data against MIA-PaCa-2;

FIG. 19 shows photocytotoxicity data against PANC-1;

FIG. 20 shows an absorption spectrum;

FIG. 21 shows a stability study spectrum.

### DETAILED DESCRIPTION

The present invention relates to the novel Chlorin e6 (Ce6)-curcumin conjugates as photosensitizers and the use for the treatment of cancers thereof.

In one aspect, the present invention relates to a novel photosensitizers, which is selected from the group consisting of the structures of Formula I, II, III and IV:

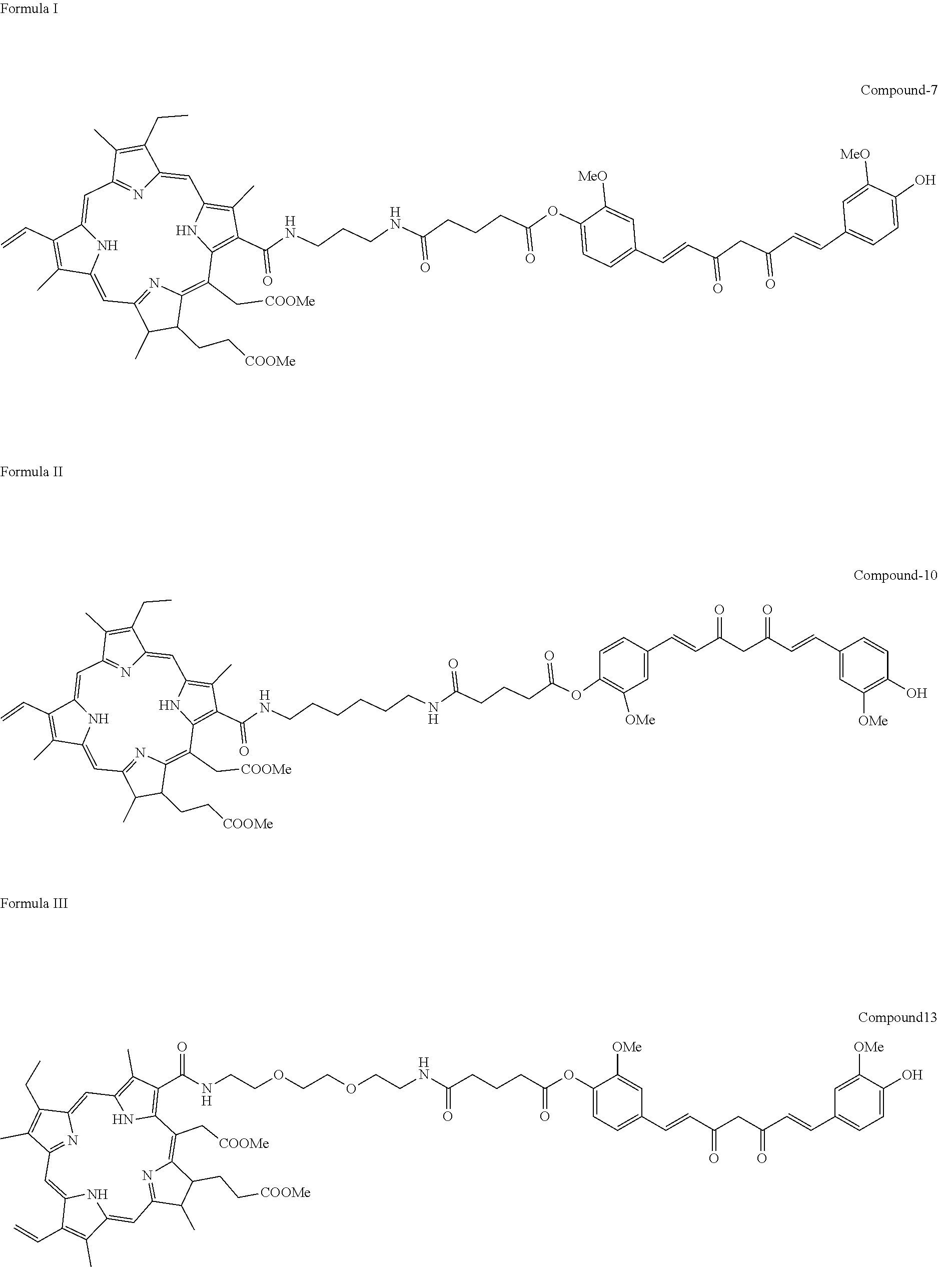 [SMILES: C=Cc1c(C)c2cc3nc(c(CC(=O)OC)c4[nH]c(cc5nc(cc1[nH]2)C(C)=C5CC)c(C)c4C(=O)NCCCNC(=O)CCCC(=O)Oc1ccc(/C=C/C(=O)CC(=O)/C=C/c2ccc(O)c(OC)c2)cc1OC)C(CCC(=O)OC)C3C ; C=Cc1c(C)c2cc3nc(c(CC(=O)OC)c4[nH]c(cc5nc(cc1[nH]2)C(C)=C5CC)c(C)c4C(=O)NCCCCCCNC(=O)CCCC(=O)Oc1ccc(/C=C/C(=O)CC(=O)/C=C/c2ccc(O)c(OC)c2)cc1OC)C(CCC(=O)OC)C3C ; C=Cc1c(C)c2cc3nc(c(CC(=O)OC)c4[nH]c(cc5nc(cc1[nH]2)C(C)=C5CC)c(C)c4C(=O)NCCOCCOCCNC(=O)CCCC(=O)Oc1ccc(/C=C/C(=O)CC(=O)/C=C/c2ccc(O)c(OC)c2)cc1OC)C(CCC(=O)OC)C3C]

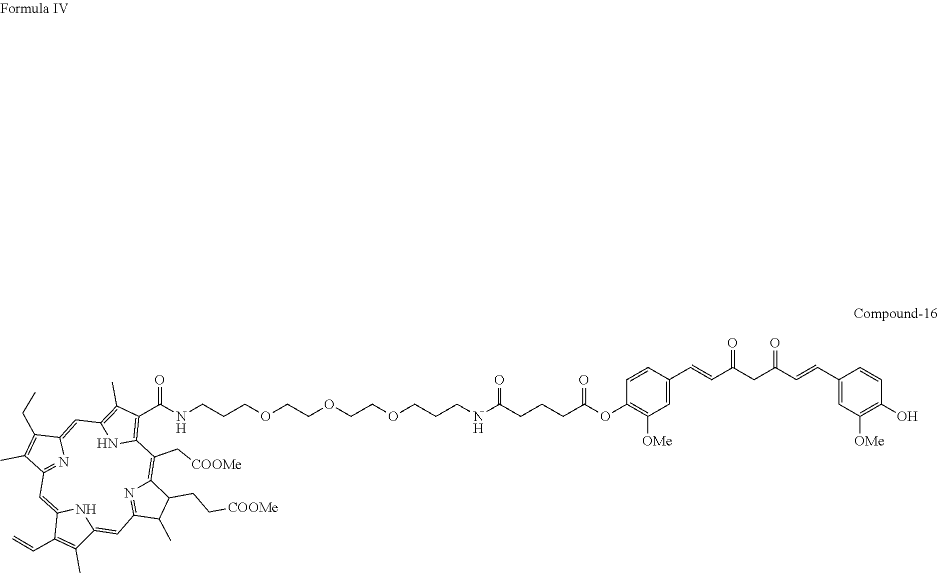 [SMILES: C=Cc1c(C)c2cc3nc(c(CC(=O)OC)c4[nH]c(cc5nc(cc1[nH]2)C(C)=C5CC)c(C)c4C(=O)NCCCOCCOCCOCCCNC(=O)CCCC(=O)Oc1ccc(/C=C/C(=O)CC(=O)/C=C/c2ccc(O)c(OC)c2)cc1OC)C(CCC(=O)OC)C3C]

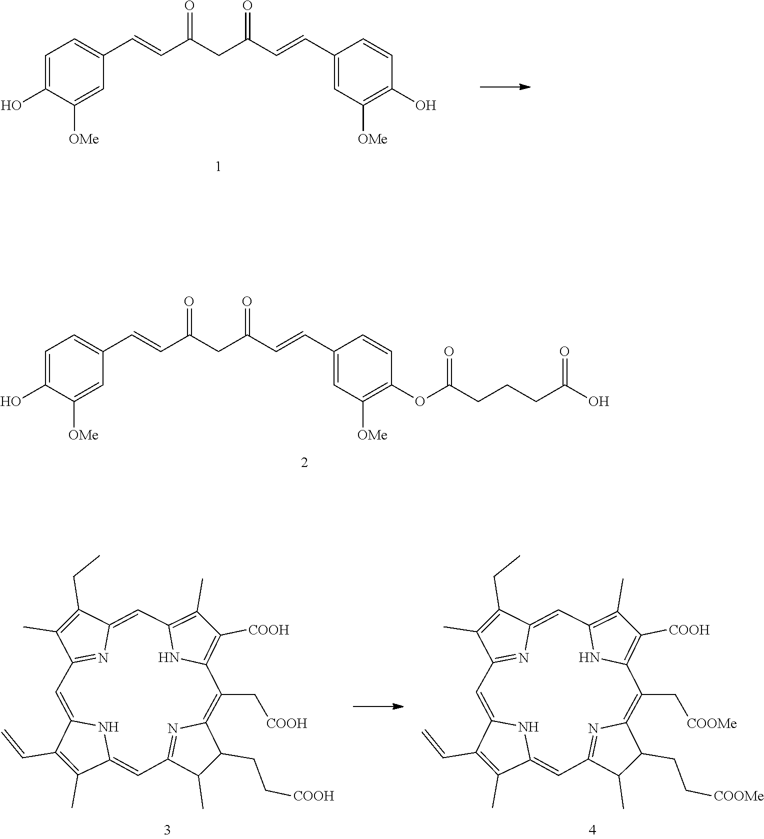 [SMILES: COc1cc(/C=C/C(=O)CC(=O)/C=C/c2ccc(O)c(OC)c2)ccc1O ; COc1cc(/C=C/C(=O)CC(=O)/C=C/c2ccc(OC(=O)CCCC(=O)O)c(OC)c2)ccc1O ; C=Cc1c(C)c2cc3nc(c(CC(=O)O)c4[nH]c(cc5nc(cc1[nH]2)C(C)=C5CC)c(C)c4C(=O)O)C(CCC(=O)O)C3C ; C=Cc1c(C)c2cc3nc(c(CC(=O)OC)c4[nH]c(cc5nc(cc1[nH]2)C(C)=C5CC)c(C)c4C(=O)O)C(CCC(=O)OC)C3C]

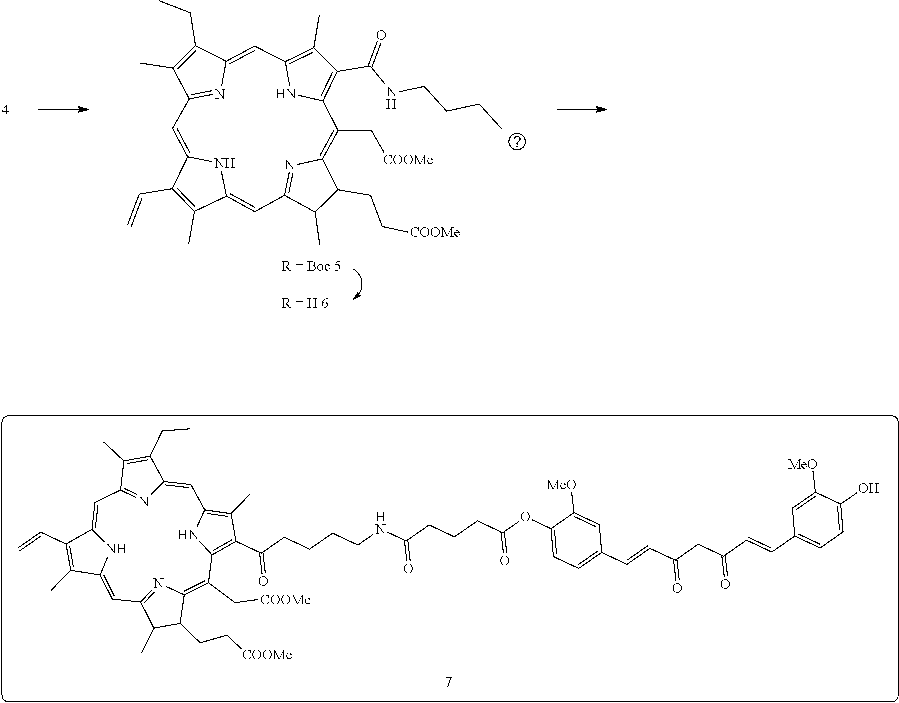 [SMILES: C=Cc1c(C)c2cc3nc(c(CC(=O)OC)c4[nH]c(cc5nc(cc1[nH]2)C(C)=C5CC)c(C)c4C(=O)NCCCC)C(CCC(=O)OC)C3C ; C=Cc1c(C)c2cc3nc(c(CC(=O)OC)c4[nH]c(cc5nc(cc1[nH]2)C(C)=C5CC)c(C)c4C(=O)CCCCNC(=O)CCCC(=O)Oc1ccc(/C=C/C(=O)CC(=O)/C=C/c2ccc(O)c(OC)c2)cc1OC)C(CCC(=O)OC)C3C]

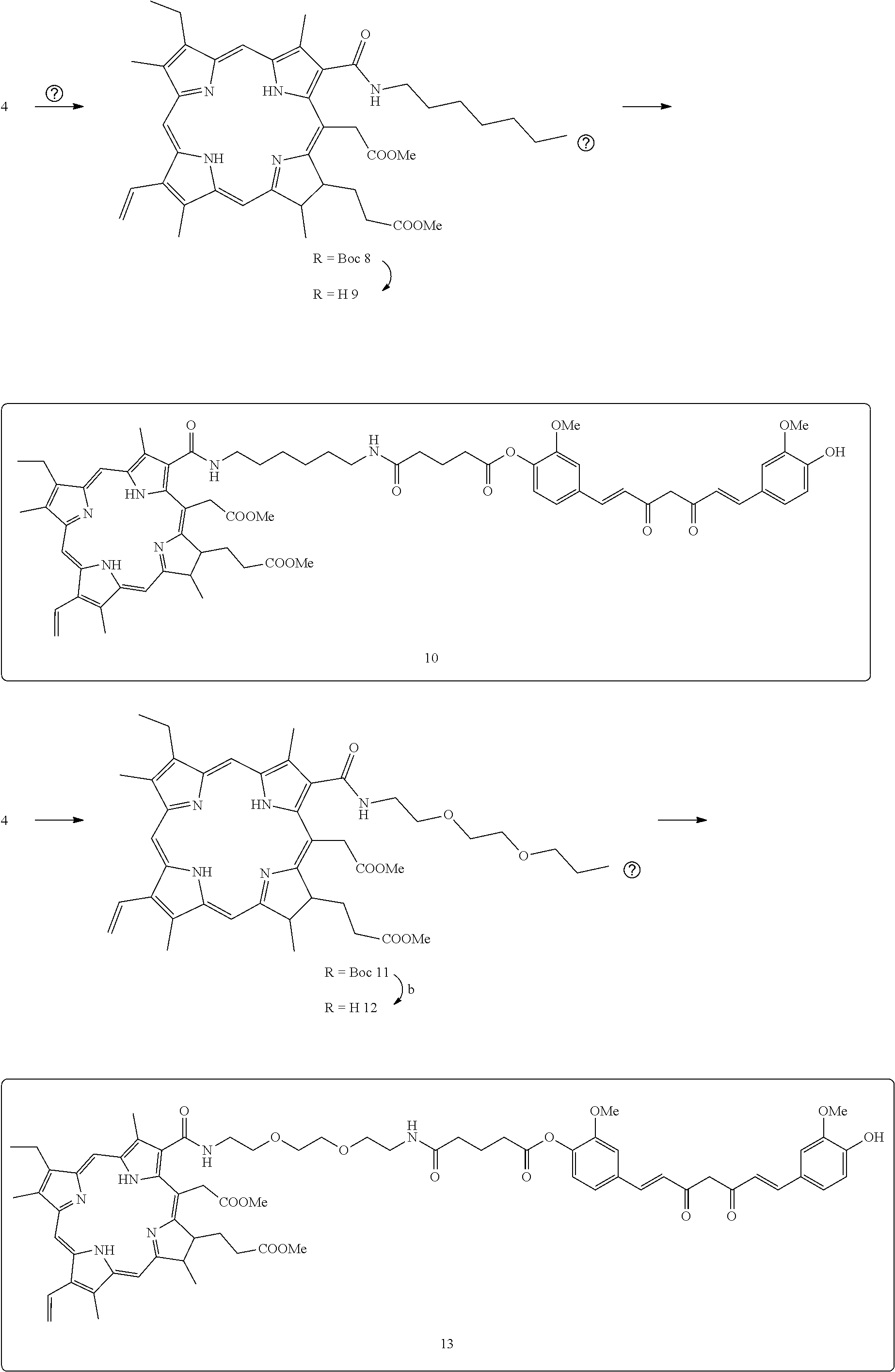

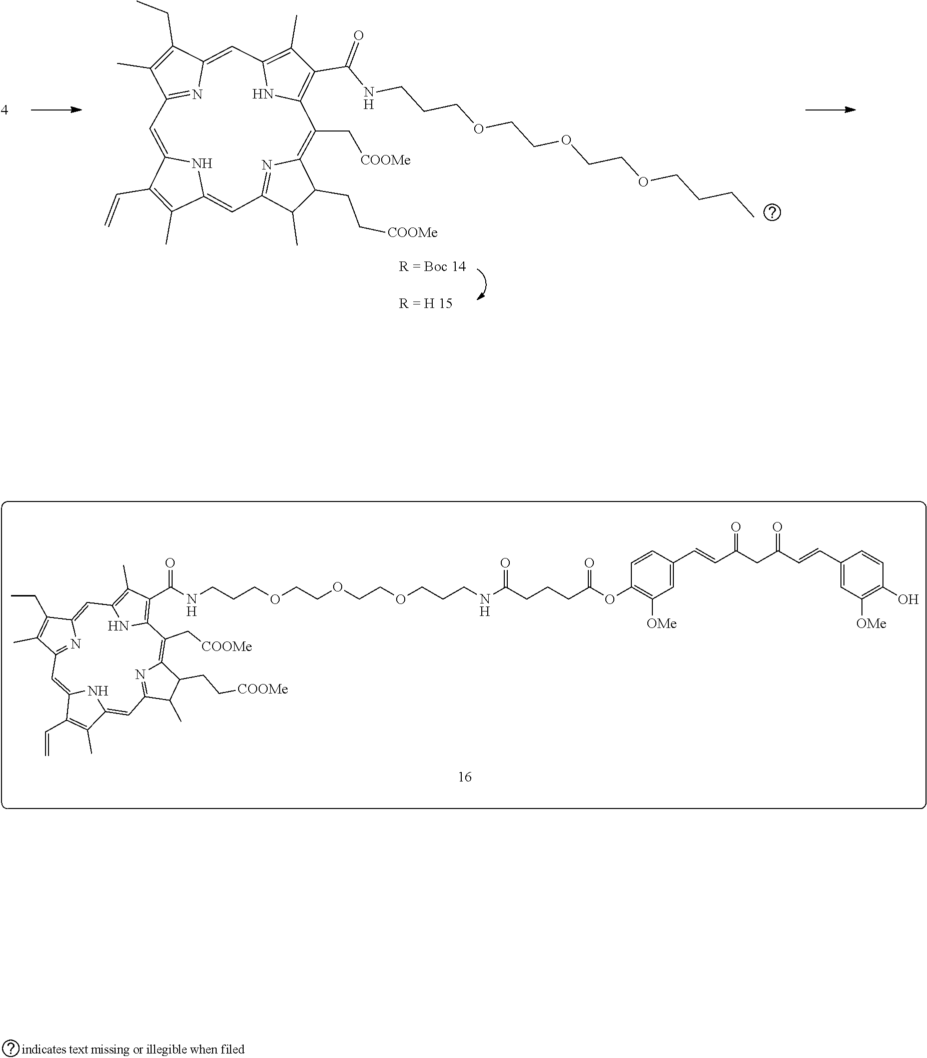

In another aspect, the present invention provides a method for preparing a novel chlorine e6-curcumin derivatives, which is represented by the following formula I-IV, comprising the steps of:

Reacting curcumin (1) with glutaric anhydride under argon atmosphere at reflux to obtain 5-(4-((1E,6E)-7-(4-hydroxy-3-methoxyphenyl)-3,5-dioxohepta-1,6-dien-1-yl)-2-methoxyphenoxy)-5-oxopentanoic acid (2);

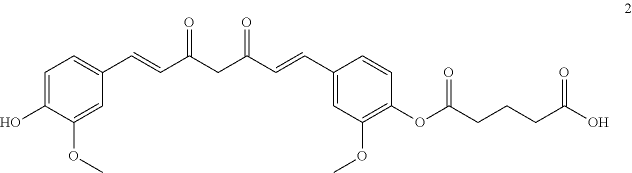 [SMILES: COc1cc(/C=C/C(=O)CC(=O)/C=C/c2ccc(OC(=O)CCCC(=O)O)c(OC)c2)ccc1O]

Reacting chlorine e6 (3) with 5% H₂SO₄ in methanol under argon atmosphere at room temperature to give dimethyl ester chlorine e6 (4);

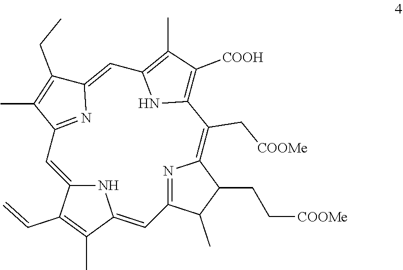 [SMILES: C=Cc1c(C)c2cc3nc(c(CC(=O)OC)c4[nH]c(cc5nc(cc1[nH]2)C(C)=C5CC)c(C)c4C(=O)O)C(CCC(=O)OC)C3C]

Reacting dimethyl ester chlorine e6 (4) with tert-butyl (3-aminopropyl)carbamate or tert-butyl (6-aminohexyl)carbamate or tert-butyl(2-(2-(2-aminoethoxy)ethoxy)ethyl)carbamate or tert-butyl (3-(2-(2-(3-aminopropoxy)ethoxy)ethoxy)propyl)carbamate under nitrogen atmosphere to obtain [tert-butyl (3-aminopropyl)carbamate]-dimethylester chlorine e6 (5) or [tert-butyl (6-aminohexyl)carbamate]-dimethylester chlorine e6 (8) or [tert-butyl(2-(2-(2-aminoethoxy)ethoxy)ethyl)carbamate]-dimethylester chlorine e6 (11) or [tert-butyl (3-(2-(2-(3-aminopropoxy)ethoxy)ethoxy)propyl)carbamate]-dimethylester chlorine e6 (14) (Scheme-1);

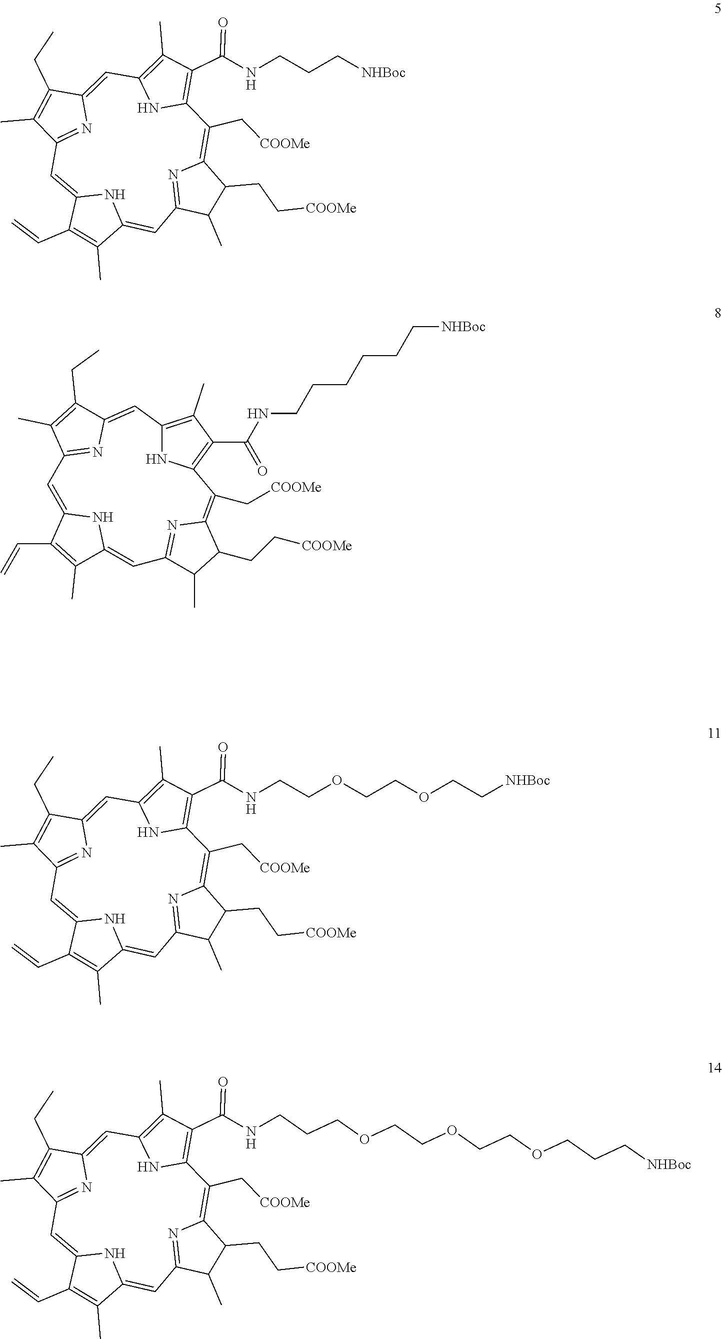 [SMILES: C=Cc1c(C)c2cc3nc(c(CC(=O)OC)c4[nH]c(cc5nc(cc1[nH]2)C(C)=C5CC)c(C)c4C(=O)NCCCNC(=O)OC(C)(C)C)C(CCC(=O)OC)C3C ; C=Cc1c(C)c2cc3nc(c(CC(=O)OC)c4[nH]c(cc5nc(cc1[nH]2)C(C)=C5CC)c(C)c4C(=O)NCCCCCCNC(=O)OC(C)(C)C)C(CCC(=O)OC)C3C ; C=Cc1c(C)c2cc3nc(c(CC(=O)OC)c4[nH]c(cc5nc(cc1[nH]2)C(C)=C5CC)c(C)c4C(=O)NCCOCCOCCNC(=O)OC(C)(C)C)C(CCC(=O)OC)C3C ; C=Cc1c(C)c2cc3nc(c(CC(=O)OC)c4[nH]c(cc5nc(cc1[nH]2)C(C)=C5CC)c(C)c4C(=O)NCCCOCCOCCOCCCNC(=O)OC(C)(C)C)C(CCC(=O)OC)C3C]

Treating [tert-butyl (3-aminopropyl)carbamate]-dimethylester chlorine e6 (5) or [tert-butyl (6-aminohexyl)carbamate]-dimethylester chlorine e6 (8) or [tert-butyl(2-(2-(2-aminoethoxy)ethoxy)ethyl)carbamate]-dimethylester chlorine e6 (11) or [tert-butyl (3-(2-(2-(3-aminopropoxy)ethoxy)ethoxy)propyl)carbamate]-dimethylester chlorine e6 (14) with Trifluoroacetic acid to give [(3-aminopropyl)carbamate]-dimethylester chlorine e6 (6) or [butyl (6-aminohexyl)carbamate]-dimethylester chlorine e6 (9) or [(2-(2-(2-aminoethoxy)ethoxy)ethyl)carbamate]-dimethylester chlorine e6 (12) or [(3-(2-(2-(3-aminopropoxy)ethoxy)ethoxy)propyl)carbamate]-dimethylester chlorine e6 (15);

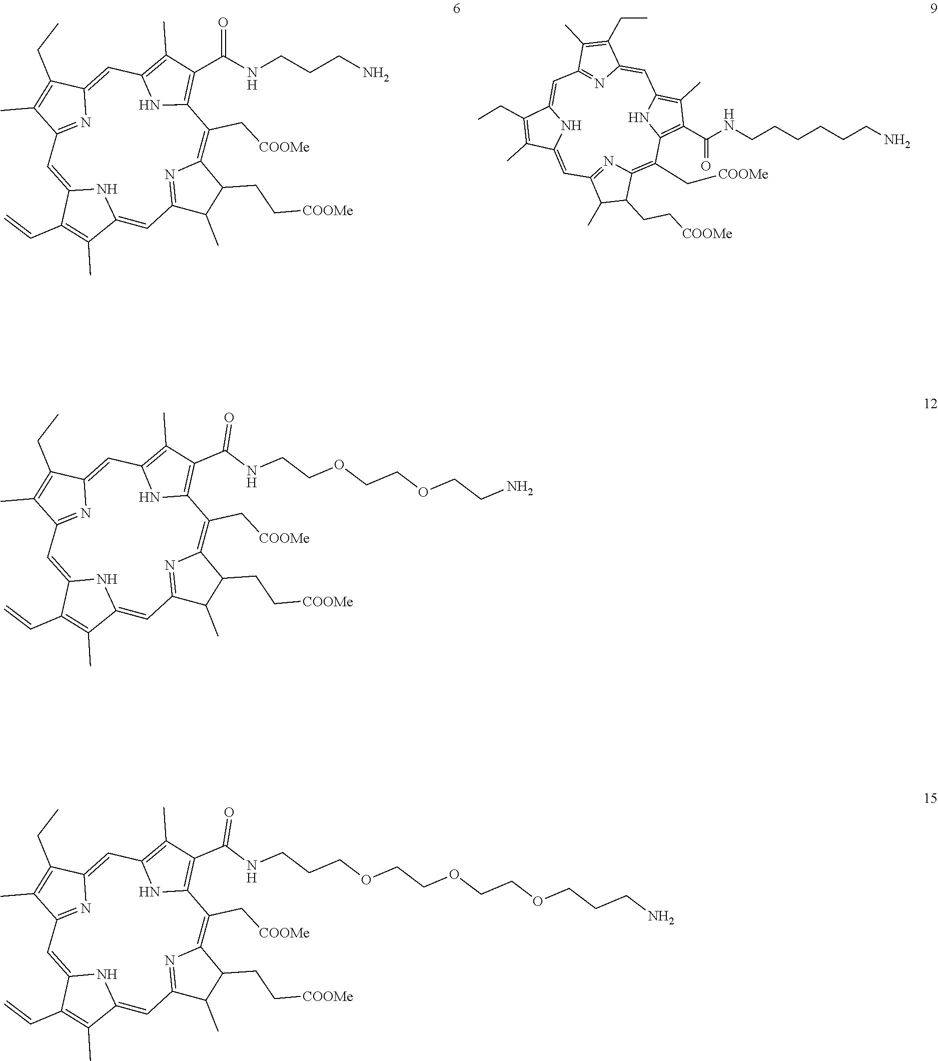 [SMILES: C=Cc1c(C)c2cc3nc(c(CC(=O)OC)c4[nH]c(cc5nc(cc1[nH]2)C(C)=C5CC)c(C)c4C(=O)NCCCN)C(CCC(=O)OC)C3C ; CCC1=C(C)c2cc3[nH]c(cc4nc(c(CC(=O)OC)c5[nH]c(cc1n2)c(C)c5C(=O)NCCCCCCN)C(CCC(=O)OC)C4C)c(C)c3CC ; C=Cc1c(C)c2cc3nc(c(CC(=O)OC)c4[nH]c(cc5nc(cc1[nH]2)C(C)=C5CC)c(C)c4C(=O)NCCOCCOCCN)C(CCC(=O)OC)C3C ; C=Cc1c(C)c2cc3nc(c(CC(=O)OC)c4[nH]c(cc5nc(cc1[nH]2)C(C)=C5CC)c(C)c4C(=O)NCCCOCCOCCOCCCN)C(CCC(=O)OC)C3C]

Final coupling of 5-(4-((1E,6E)-7-(4-hydroxy-3-methoxyphenyl)-3,5-dioxohepta-1,6-dien-1-yl)-2-methoxyphenoxy)-5-oxopentanoic acid (2) and [(3-aminopropyl)carbamate]-dimethylester chlorine e6 (6) or [butyl (6-aminohexyl)carbamate]-dimethylester chlorine e6 (9) or [(2-(2-(2-aminoethoxy)ethoxy)ethyl)carbamate]-dimethylester chlorine e6 (12) or [(3-(2-(2-(3-aminopropoxy)ethoxy)ethoxy)propyl)carbamate]-dimethylester chlorine e6 (15) give the chlorine e6-curcumin derivatives of Formula I-IV.

In more specific embodiments, the invention relates to four photo sensitizers compound 7, 10, 13, 16 (Formula IV), with four different linkers, two hydrophobic and two hydrophilic linkers to conjugate chlorine e6 and curcumin (scheme-1).

The curcumin butanoic acid 2 is synthesized by reacting curcumin 1 with glutaric anhydride in the presence of base.

Dimethyl chlorine e6 4 was synthesized from chlorin e6 and 5% H₂SO₄ in methanol. Here after chlorine e6 was denoted as Ce6.

Dimethyl Ce6 was conjugated with tert-butyl (3-aminopropyl)carbamate using EDCl, HOBt as a coupling agent, in CHCl₃ at room temperature to give compound 5.

Dimethyl Ce6 was conjugated with tert-butyl (6-aminohexyl)carbamate using EDCl, HOBt as a coupling agent, in CHCl₃ at room temperature to give compound 8.

Dimethyl Ce6 was conjugated with tert-butyl(2-(2-(2-aminoethoxy) ethoxy)ethyl)carbamate using EDCl, HOBt as a coupling agent, in CHCl₃ at room temperature to give compound 11.

Dimethyl Ce6 was conjugated with tert-butyl (3-(2-(2-(3-aminopropoxy) ethoxy)ethoxy)propyl)carbamate using EDCl, HOBt as a coupling agent, in CHCl₃ at room temperature to give compound 14.

Compound 5, 8, 11, 14 were reacted with trifluoroacetic acid in CHCl₃ at room temperature gave compound 6, 9, 12, 15.

The photosensitizers 7, 10, 13, and 16 (Formula IV) were prepared by coupling compound 6, 9, 12, 15 with compound 2 using EDCl, HOBt as a coupling agent, in CHCl₃ at room temperature.

The photophysical properties of Ce6-curcumin conjugates and dimethyl ester ce6 were analyzed in dimethyl sulfoxide (DMSO). All the photosensitizers 7, 10, 13, and 16 (Formula IV) effectively absorbed the red light compared to dimethyl ester ce6. The major soret peak at λmax=405-408 nm and Q-bands at 667 nm for the photosensitizers 7, 10, 13, and 16 (Formula IV) and Q band peak at 656 for dimethyl ester ce6. The lowest energy Q-band of ce6-curcumin derivatives showed red-shift by 11 nm (figure).

The stability study of Ce6-curcumin derivatives was examined in PBS (Figure) the stability was measured by UV-Visible spectrophotometer at room temperature. The present invention compounds (7, 10, 13, and 16 Formula IV) have shown stability up to 60 h and it was confirmed by LC-MS.

In the present invention, in vitro therapeutic effect PDT was compared among the photosensitizers, 7, 10, 13, 16, and 3 in an in vitro light-induced cytotoxicity test.

Dark cytotoxicity: The culture of AsPC-1 pancreatic cancer cells were grown in RPMI-1640 medium (life technologies corporation, USA) supplemented with 10% heat-inactivated fetal bovine serum (life technologies corporation, USA) and 1 penicillin (life technologies corporation, USA), whereas MIA-paca2 and PANC-1 cells were grown in DMEM. The MTT assay was used to assess the cell viability of AsPC-1, MIA-paca2, PANC-1 cells. The cells attached to a 96-well plate (5,000 cells/well) were treated with 3.125 μM, 6.25 μM, 12.5 μM, 25 μM, 50 μM of photosensitizers, 7, 10, 13, 16, and 3 for 3 h. The cells were then incubated for 72 h at 37° C. in a 5% CO₂ incubator and exposed to MTT [3-(4,5-dimethylthiazol-2-yl)-2,5-diphenyl-tetrazolium bromide] (250 μg/ml) for 3 h. The solution was changed to 200 μl of dimethylsulfoxide (DMSO, Sigma). After 30 min incubation and shaking in microplate mixer, the optical density (OD) was measured using a microplate reader (Thermo Fisher scientific, USA) at 570 nm wavelength. The cell viability was calculated using the following formula: Cell viability (%)=Mean optical density of treated wells/Mean optical density of control wells×100.

Phototoxicity: The culture of AsPC-1 pancreatic cancer cells were grown in RPMI-1640 medium (life technologies corporation, USA) supplemented with 10% heat-inactivated fetal bovine serum (life technologies corporation, USA) and 1% penicillin (life technologies corporation, USA), whereas MIA-paca2 and PANC-1 cells were grown in DMEM. The MTT assay was used to assess the cell viability of AsPC-1, MIA-paca2, PANC-1 cells. The cells attached to a 96-well plate (5,000 cells/well) were treated with 3.125 μM, 6.25 μM, 12.5 μM, 25 μM, 50 μM of photosensitizers, 7, 10, 13, 16, and 3 for 3 h. The photosensitized cells were then irradiated with a 50 mW, 0.09 J/cm² laser for 100 sec. The cells were then incubated for 72 h at 37° C. in a 5% CO₂ incubator and exposed to MTT [3-(4,5-dimethylthiazol-2-yl)-2,5-diphenyl-tetrazolium bromide] (250 μg/ml) for 3 h. The solution was changed to 200 μl of dimethylsulfoxide (DMSO, Sigma). After 30 min incubation and shaking in microplate mixer, the optical density (OD) was measured using a microplate reader (Thermo Fisher scientific, USA) at 570 nm wavelength. The cell viability was calculated using the following formula: Cell viability (%)=Mean optical density of treated wells/Mean optical density of control wells×100.

TABLE 1
Dark Cytotoxicity against AsPC-1

| Photo- | | | | | |
|---|---|---|---|---|---|
| sensitizer | Com- | Com- | Com- | Com- | Com- |
| Conc | pound | pound | pound | pound | pound |
| (μM) | 7 | 10 | 13 | 16 | 3 |
| 0 | 100.00 | 100.00 | 100.00 | 100.00 | 100.00 |
| 3.12 | 100.58 | 96.52 | 105.36 | 99.87 | 98.78 |
| 6.25 | 101.61 | 98.21 | 107.93 | 102.84 | 101.11 |
| 12.5 | 103.72 | 98.52 | 106.07 | 102.84 | 100.36 |
| 25 | 97.17 | 95.05 | 98.45 | 80.37 | 99.61 |
| 50 | 90.14 | 78.85 | 78.73 | 84.12 | 100.23 |

TABLE 2
Dark cytotoxicity against MIA-PaCa-2

| Photo- | | | | | |
|---|---|---|---|---|---|
| sensitizer | Com- | Com- | Com- | Com- | Com- |
| Conc | pound | pound | pound | pound | pound |
| (μM) | 7 | 10 | 13 | 16 | 3 |
| 0 | 100.00 | 100.00 | 100.00 | 100.00 | 100.00 |
| 3.12 | 107.39 | 89.66 | 113.68 | 121.98 | 98.78 |
| 6.25 | 104.17 | 82.04 | 114.89 | 121.14 | 101.11 |
| 12.5 | 101.89 | 75.11 | 114.50 | 113.19 | 100.36 |
| 25 | 99.23 | 66.44 | 96.27 | 100.97 | 99.61 |
| 50 | 88.84 | 48.71 | 66.78 | 72.84 | 100.23 |

TABLE 3
Phototoxicity against AsPC-1

| Photo- | | | | | |
|---|---|---|---|---|---|
| sensitizer | Com- | Com- | Com- | Com- | Com- |
| Conc | pound | pound | pound | pound | pound |
| (μM) | 7 | 10 | 13 | 16 | 3 |
| 0 | 100 | 100.00 | 100 | 100 | 100 |
| 3.12 | 85.97 | 85.90 | 7.34 | 87.29 | 91.05 |
| 6.25 | 43.03 | 75.12 | 6.61 | 45.79 | 76.65 |
| 12.5 | 24.03 | 34.05 | 6.48 | 32.67 | 66.31 |
| 25 | 10.35 | 26.11 | 6.58 | 25.46 | 37.32 |
| 50 | 6.63 | 23.17 | 6.58 | 27.83 | 17.52 |

TABLE 4
Phototoxicity against MIA-PaCa-2

| Photo- | | | | |
|---|---|---|---|---|
| sensitizer | Com- | Com- | Com- | Com- |
| Conc | pound | pound | pound | pound |
| (μM) | 7 | 10 | 13 | 16 |
| 0 | 100 | 100 | 100 | 100 |
| 3.12 | 69.70 | 94.15 | 4.68 | 80.67 |
| 6.25 | 12.35 | 90.03 | 5.07 | 17.62 |
| 12.5 | 6.03 | 85.20 | 4.93 | 9.49 |
| 25 | 5.18 | 64.88 | 5.39 | 8.03 |
| 50 | 4.99 | 48.21 | 5.90 | 9.67 |

TABLE 5
Phototoxicity against PANC-1

| Photo- | | | | | |
|---|---|---|---|---|---|
| sensitizer | Com- | Com- | Com- | Com- | Com- |
| Conc | pound | pound | pound | pound | pound |
| (μM) | 7 | 10 | 13 | 16 | 3 |
| 0 | 100 | 100.00 | 100 | 100 | 100 |
| 3.12 | 66.14 | 99.74 | 8.51 | 97.36 | 104.37 |
| 6.25 | 31.56 | 96.76 | 7.71 | 89.19 | 82.85 |
| 12.5 | 12.33 | 81.89 | 7.53 | 59.07 | 30.03 |
| 25 | 11.58 | 81.07 | 8.04 | 24.07 | 8.57 |
| 50 | 11.87 | 73.00 | 7.28 | 20.24 | 7.61 |

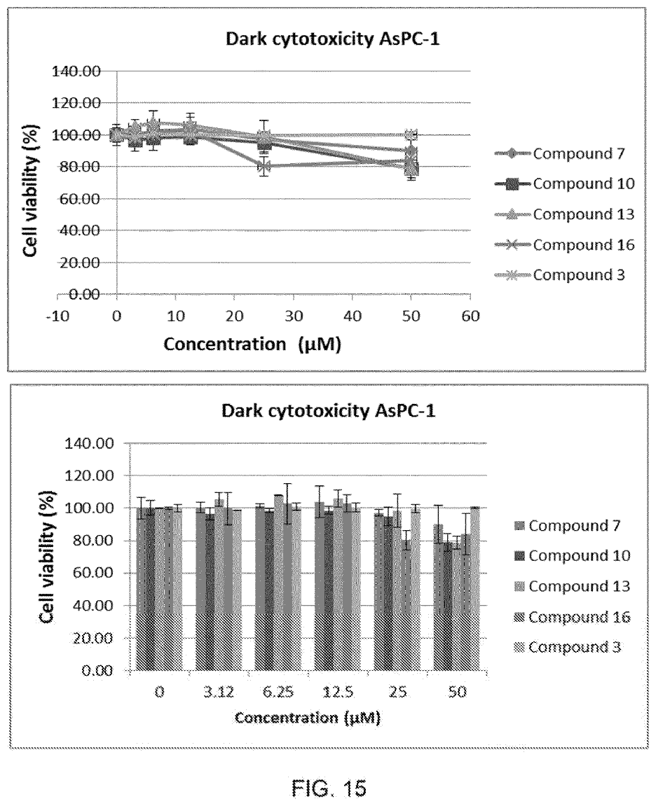

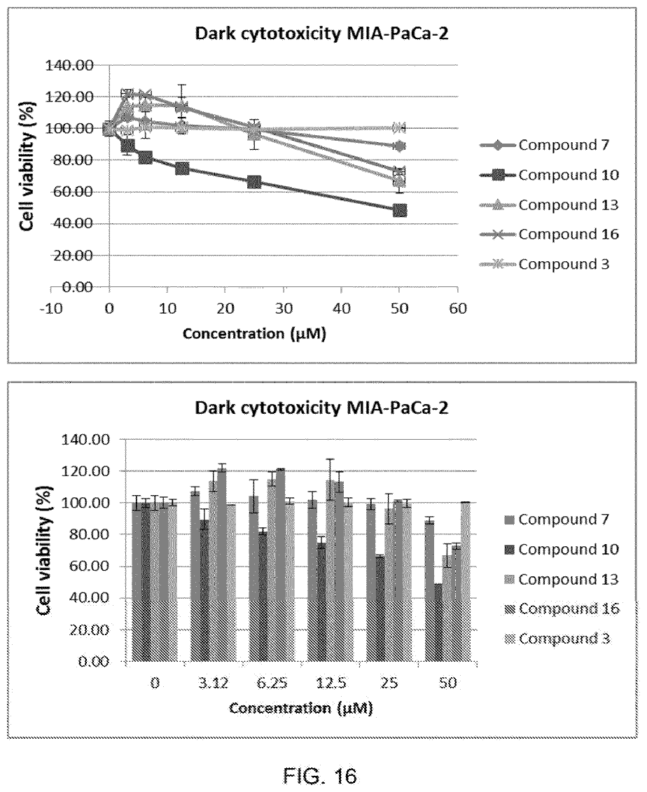

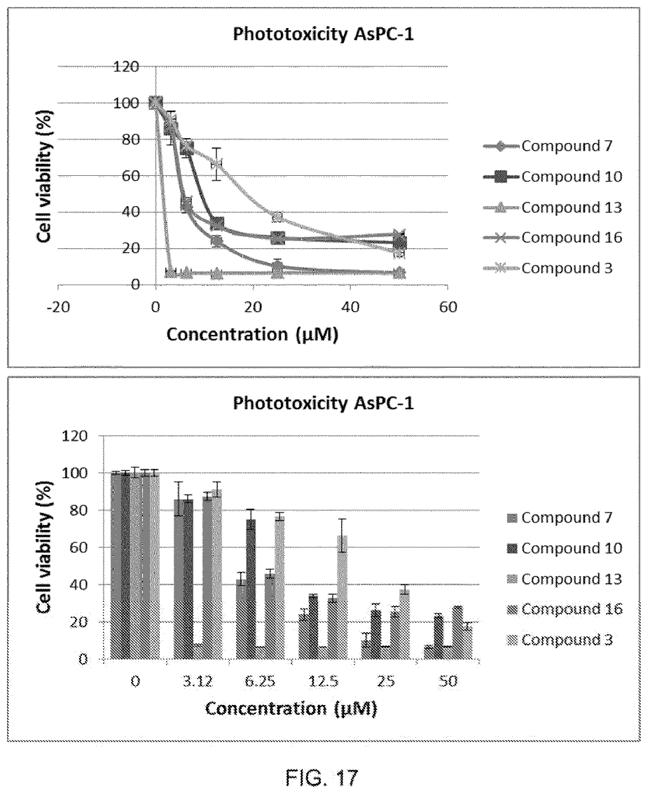

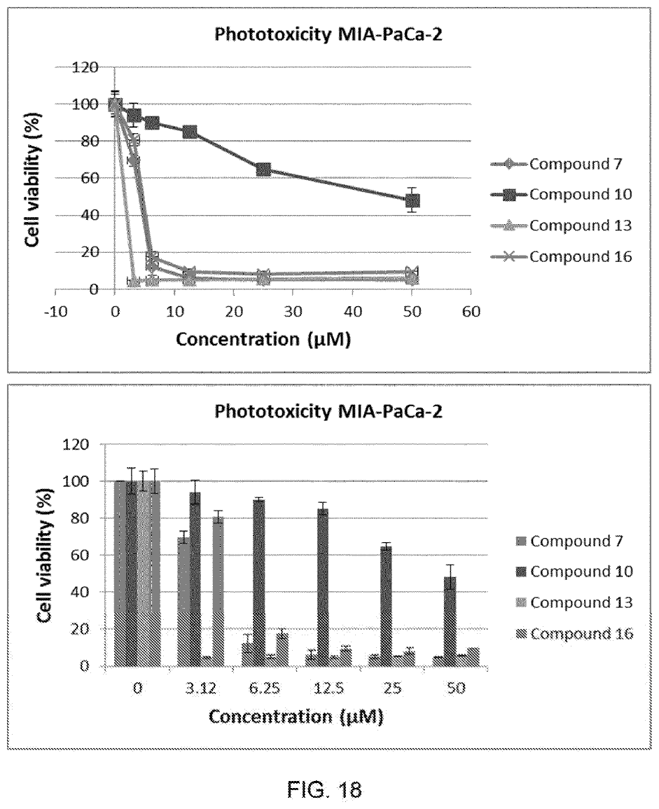

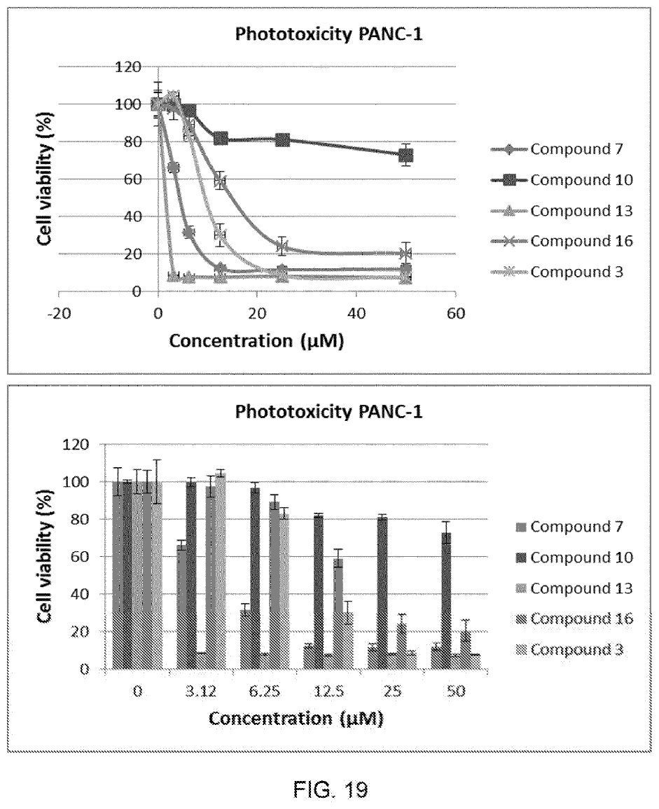

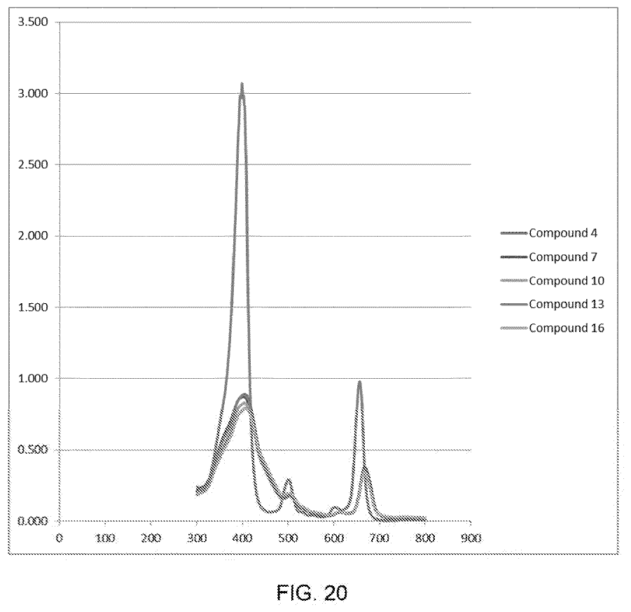

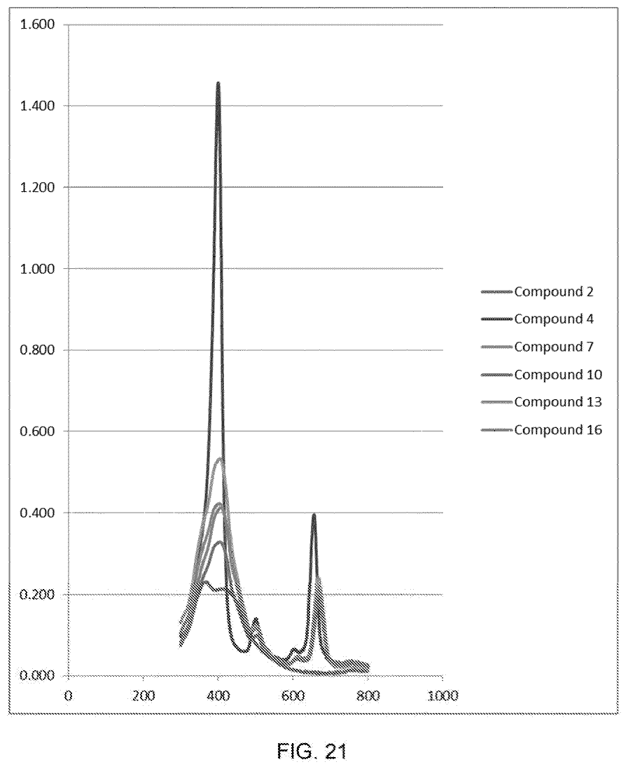

As shown in Table 1-2, all the compounds of present invention were treated with a different concentration in AsPC-1 and MIA-PaCa-2 cell lines were incubated for 72 h. They showed less cytotoxicity in the absence of light (FIGS. 15 & 16). From the Table 3-5 revealed that the compounds of the present invention have shown superior PDT efficacy than the Ce6 at the tested concentration. Compound 13 inhibited the AsPC-1 MIA-PaCa-2 and PANC-1 more than 90% at 3 μM concentration. Compound 7 inhibited 75, 94, and 88% of AsPC-1 MIA-PaCa-2 and PANC-1 respectively at 12.5 μM concentration. Compound 16 inhibited 75, 92, and 76% of AsPC-1 MIA-PaCa-2 and PANC-1 respectively at 25 μM concentration. Compound 3 inhibited 63 and 91% of AsPC-1, PANC-1 respectively at 25 μM concentration (FIG. 18-20).

### EXAMPLES

### Synthesis of 5-(4-((1E,6E)-7-(4-hydroxy-3-methoxyphenyl)-3,5-dioxohepta-1,6-dien-1-yl)-2-methoxyphenoxy)-5-oxopentanoic acid (2)

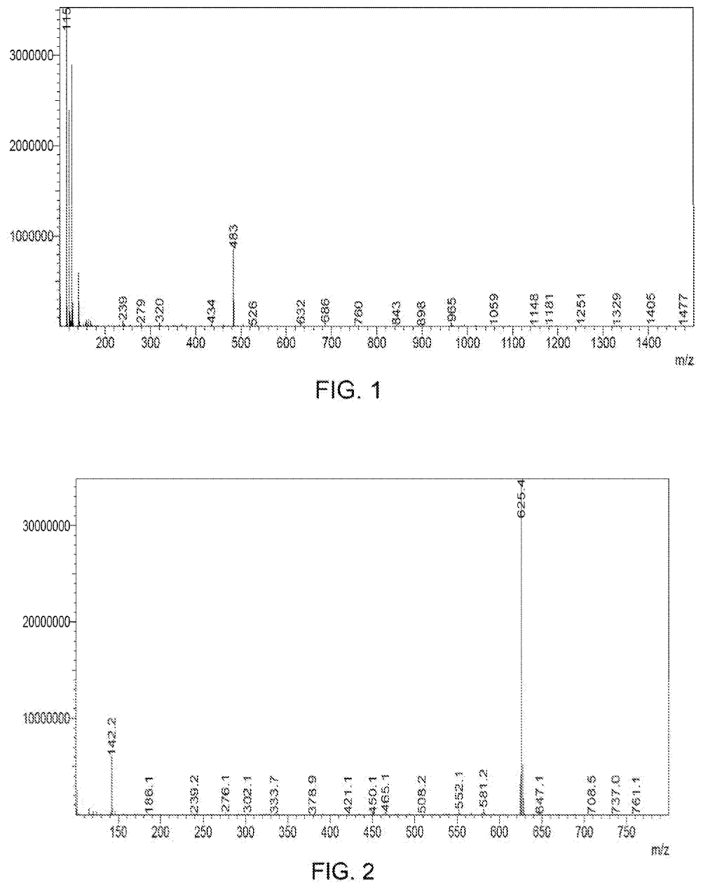

To a solution of (2.01 g, 5.46 mmol) of curcumin, and (112 mg, 0.92 mmol) of DMAP in 100 mL THF was added (1.33 mL, 9.55 mmol) of Et₃N. (0.685 g, 6 mmol) of glutaric anhydride (95%) in 5 mL THF was added slowly dropwise to the curcumin solution. The mixture was stirred and refluxed under argon overnight. THF was removed under vacuum, 55 mL EtOAc was added, followed by the addition of 15 mL of 1M HCl, the mixture was stirred for 10 minutes. The organic phase was separated and extracted with EtOAc three times; the solvent was removed and dried. The product was purified via column chromatography, eluting with CH₂Cl₂: MeOH, 95:5. Yield: 69%. ¹HNMR (CDCl₃, 400 MHz): δ 7.65 (d, J=16 Hz, 2H), 7.20-6.95 (m, 5H), 6.96 (d, 1H), 6.48-6.57 (m, 2H), 5.85 (s, 2H), 3.98 (s, 3H), 3.90 (s, 3H), 2.75-2.71 (t, J=8 Hz, 2H), 2.61-2.57 (t, J=8 Hz, 2H), 2.15-2.12 (t, J=8 Hz, 2H). ¹³C NMR (CDCl₃, 100 MHz): δ 184.56, 181.80, 178.26, 170.84, 151.28, 148.03, 146.84, 141.09, 139.40, 134.12, 127.53, 124.25, 123.07, 121.73, 120.99, 114.89, 111.37, 109.69, 101.58, 55.96, 32.82, 19.92. LC-MS: 483 [M+H] (FIG. 1).

### Synthesis of Dimethylester of Chlorine e6 (4)

Chlorin e6 (3) (3 g, 5.02 mmol) was dissolved in 5% sulfuric acid and methanol and allowed to stir protected from light, under argon overnight. The reaction was poured into cold saturated aqueous NaHCO₃ and extracted twice with CH₂Cl₂. The extract was washed twice with brine, dried over Na₂SO₄ and filtered. The solvent was evaporated. It was then purified on a silica gel column afford 2.8 g, Yield: 88%. UV-Vis (DMSO): λmax 656, 501, 399 nm. ¹H NMR (CDCl₃, 400 MHz): δ 9.62 (s, 1H), 9.49 (s, 1H), 8.73 (s, 1H), 8.03 (m, 1H), 6.32 (dd, J=17.8, 1.2 Hz, 1H), 6.13 (dd, J=11.5, 1.2 Hz, 1H), 5.50 (d, J=18.6 Hz, 1H), 5.23 (d, J=18.6 Hz, 1H), 4.45 (m, 2H), 3.82 (s, 3H), 3.76 (q, J=7.6 Hz, 2H), 3.62 (s, 6H), 3.46 (s, 3H), 3.25 (s, 3H), 1.69 and 2.12 (m, 2H), 2.19 and 2.56 (m, 2H), 1.81 (d, J=7.1 Hz, 3H), 1.64 (t, J=7.6 Hz, 3H), −1.71 (s, 1H), −1.92 (s, 1H). ¹³C NMR (CDCl₃, 100 MHz): δ 173.58, 169.89, 167.31, 155.01, 148.84, 145.12, 139.77, 137.17, 136.17, 135.85, 135.58, 134.84, 130.67, 129.33, 121.80, 102.47, 98.63, 93.60, 52.84, 51.65, 49.53, 39.22, 30.98, 29.34, 22.75, 19.59, 17.66, 12.69, 12.15, 11.29. LC-MS: 625 [M+H] (FIG. 2).

### Synthesis of tert-butyl (3-aminopropyl)carbamate

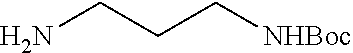 [SMILES: CC(C)(C)OC(=O)NCCCN]

To a stirred and cooled solution (0° C.) of 1, 3-diaminopropane (3.64 mL, 43.5 mmol) in CHCl₃ (45 mL) was added a solution of di-tert-butyl bicarbonate (0.95 g, 4.35 mmol) in CHCl₃ (22 mL) dropwise over a period of 3 h. The reaction mixture was allowed to warm to room temperature and stirred for additional 20 h. The precipitated white solid was filtered and the CHCl₃ was washed with water (2×20 mL). The organic layer was dried over Na₂SO₄ and concentrated in vacuo to give compound tert-butyl (3-aminopropyl)carbamate 475 mg, Yield: 63% as a clear oil which was used for the next reaction without any further purification. ¹H NMR (CDCl₃, 400 MHz): δ 4.91 (bs, 1H), 3.16 (dq, J=12.7, 6.4 Hz, 2H), 2.73 (t, J=6.6 Hz, 2H), 1.58 (p, J=6.6 Hz, 2H), 1.41 (s, 9H).

### Synthesis of Ce6-Propnane-NHBoc Conjugate (5)

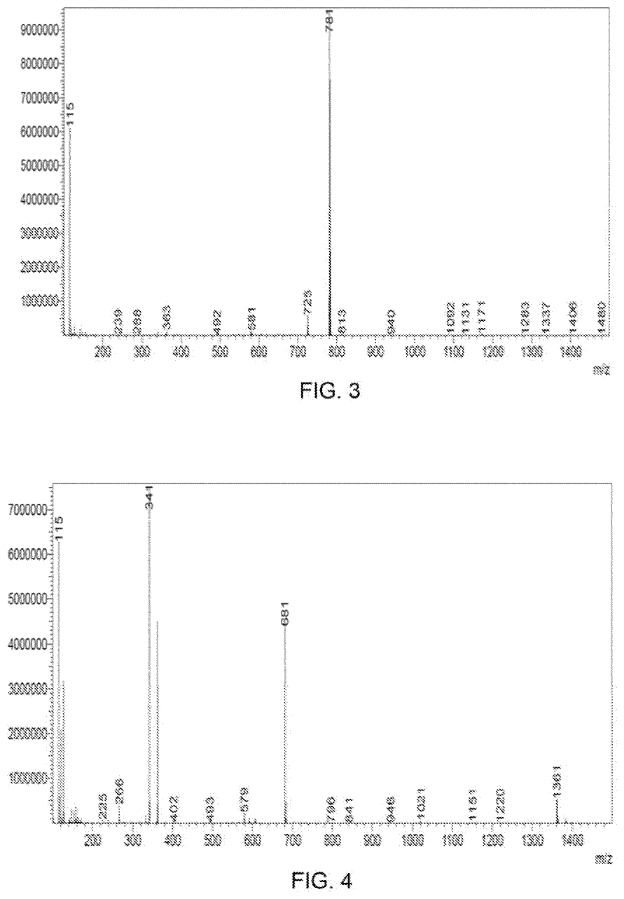

Dimethyl ester of chlorin e6 4 (1 g, 1.60 mmol) was dissolved in anhydrous CH₂Cl₂ (30 mL). EDCl (368 mg, 1.92 mmol) and HOBt (260 mg, 1.92 mmol) were then added and allowed to stir until completely dissolved under nitrogen. After 30 min, tert-butyl (3-aminopropyl)carbamate (836 mg, 4.80 mmol) and DIPEA (413 mg, 3.2 mmol) were mixed in CH₂Cl₂ (20 mL) and added to the reaction mixture. The mixture was allowed to stir at room temperature for 12 h under nitrogen. The reaction mixture was diluted with CH₂Cl₂ (200 mL) and then washed with brine and water, respectively. The organic layer was dried over anhydrous Na₂SO₄ and then evaporated. The product was purified via column chromatography to afford 520 mg of 5, Yield: 41%. UV-Vis (DMSO): λmax 670, 504, 408 nm. ¹H NMR (CDCl₃, 400 MHz): δ 9.62 (s, 1H), 9.57 (s, 1H), 8.73 (s, 1H), 8.03 (m, 1H), 6.31 (dd, J=17.8, 1.2 Hz, 1H), 6.08 (dd, J=11.5, 1.2 Hz, 1H), 5.48 (d, J=18.6 Hz, 1H), 5.20 (d, J=18.6 Hz, 1H), 4.38 and 4.27 (m, 2H), 3.76 (m, 5H), 3.58 (m, 8H), 3.40 (s, 6H), 3.25 (s, 4H), 1.69 and 2.12 (m, 2H), 1.90 and 2.49 (m, 2H), 1.90 (m, 3H), 1.68 (m, 3H) 1.50 (s, 11H), −1.67 (s, 1H), −1.88 (s, 1H). ¹³C NMR (CDCl₃, 100 MHz): δ 173.87, 168.78, 166.70, 156.56, 154.17, 149.07, 144.74, 138.86, 136.10, 13503, 134.77, 134.50, 130.14, 129.90, 129.45, 128.20, 121.61, 102.15, 101.40, 98.83, 93.67, 79.35, 53.08, 52.17, 51.65, 49.26, 37.62, 31.14, 30.37, 29.64, 28.38, 23.05, 19.69, 17.76, 12.19, 11.35. LC-MS: 781 [M+H] (FIG. 3).

### Synthesis of Ce6-Propnane Amine Conjugate (6)

The compound 5 (500 mg, 0.64 mmol) was dissolved in of dry CH₂Cl₂ (20 mL) in an ice bath under argon. TFA (2 mL) was added, and the reaction mixture was stirred overnight. The reaction mixture was evaporated several times with diethyl ether to remove residual TFA. Then the precipitate was dissolved in CH₂Cl₂ and washed three times with H₂O and once with 10% NaHCO₃ to remove TFA. The organic layer was dried over anhydrous Na₂SO₄ and then evaporated to give a crude compound, purified by silica gel chromatography to give 350 mg of 6, Yield: 80%. UV-Vis (DMSO): λmax 658, 501, 400 nm. ¹H NMR (CDCl₃, 400 MHz): δ 9.62 (s, 1H), 9.56 (s, 1H), 8.73 (s, 1H), 8.01 (m, 1H), 6.29 (dd, J=16 Hz, 1H), 6.07 (dd, J=16 Hz, 1H), 5.50 (d, J=20 Hz, 1H), 5.20 (d, J=20 Hz, 1H), 4.40 and 4.27 (m, 2H), 3.86 and 3.61 (m, 2H), 3.71 (m, 5H), 3.53 (s, 3H), 3.48 (s, 4H), 3.41 (s, 3H), 3.26 (s, 3H), 2.89 (t, J=8 Hz, 2H), 2.49 (m, 2H), 1.69 and 2.12 (m, 2H), 1.85 (t, J=4 & 8 Hz, 2H), 1.77 (m, 6H), −1.71 (s, 1H), −1.92 (s, 1H); ¹³C NMR (CDCl₃, 100 MHz): δ 173.87, 168.78, 166.70, 154.17, 149.07, 144.74, 138.86, 136.10, 135.03, 134.77, 134.50, 130.14, 129.90, 129.45, 128.20, 121.61, 102.15, 101.40, 98.83, 93.67, 53.08, 52.17, 51.65, 49.26, 37.62, 31.14, 30.37, 29.64, 28.38, 23.05, 19.69, 17.76, 12.19, 11.35. LC-MS: 681 [M+H] (FIG. 4).

### Synthesis of Chlorine e6-curcumin Conjugate (7)

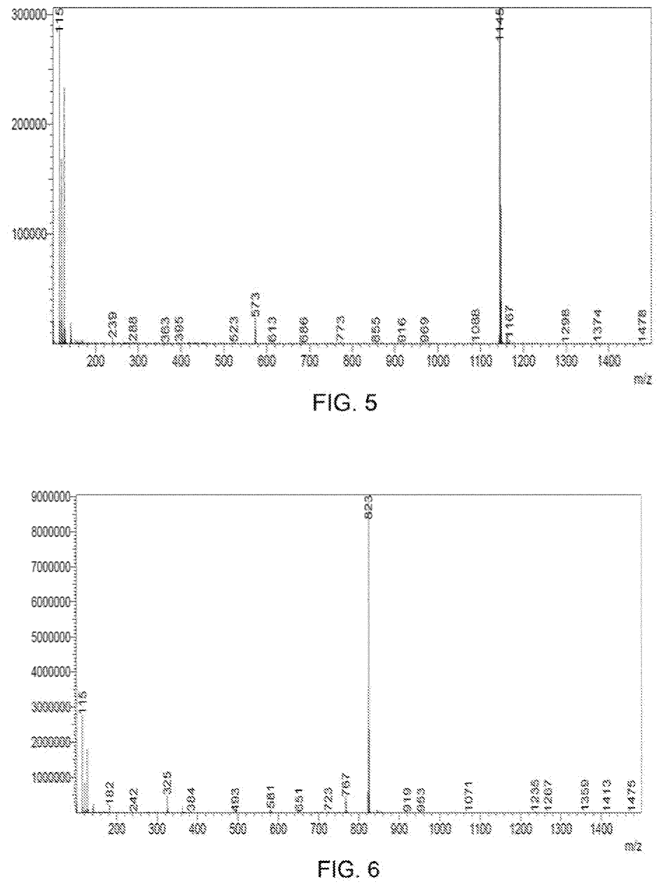

Compound 2 (250 mg, 0.51 mmol) was dissolved in dry CH₂Cl₂. A mixture of HOBt (83 mg, 0.62 mmol), EDCl (120 mg, 0.62 mmol), and DIPEA (66 mg, 0.51 mmol) in CH₂Cl₂ was added, and the mixture was allowed to stir for 30 min. Compound 6 (352 mg, 0.51 mmol) and DIEA (66 mg, 0.51 mmol) were mixed in CH₂Cl₂ and added to this reaction mixture. The mixture was stirred overnight. It was diluted with CH₂Cl₂ and then washed with 5% aqueous citric acid, followed by a wash with brine and water. It was dried over anhydrous Na₂SO₄ and then evaporated. The residue was purified by silica gel column chromatography to afford 280 mg of 7, Yield: 47%. UV-Vis (DMSO): λmax 668, 504, 403 nm. ¹H NMR (CDCl₃, 400 MHz): δ 9.59 (s, 1H), 9.55 (s, 1H), 8.72 (s, 1H), 8.00 (m, 1H), 7.49 (d, J=16 Hz, 1H), 7.40 (d, J=16 Hz, 1H), 7.01 (m, 2H), 6.93 (m, 4H), 6.85-6.81 (m, 2H), 6.33-6.25 (m, 2H), 6.07 (dd, J=4 Hz, 1H), 5.56 (s, 1H), 5.44 (d, J=16 Hz, 1H), 5.19 (d, J=20 Hz, 1H), 4.40 and 4.26 (m, 2H), 3.85 (m, 5H), 3.75-3.69 (m, 8H), 3.57-3.53 (m, 6H), 3.46-3.40 (m, 8H), 3.23 (s, 3H), 2.58 (t, J=8 Hz, 2H), 2.50 (m, 1H), 2.34 (t, J=8 & 4 Hz, 2H), 2.15-2.09 (m, 2H), 2.05 (t, J=8 Hz, 2H), 1.67 (m, 6H), −1.71 (s, 1H), −1.92 (s, 1H). ¹³C NMR (CDCl₃, 100 MHz): δ 184.43, 181.53, 173.59, 172.69, 171.19, 170.04, 168.90, 166.73, 151.17, 149.03, 147.92, 146.75, 144.81, 141.01, 140.94, 139.13, 136.17, 134.92, 134.85, 134.60, 134.56, 133.94, 130.27, 129.77, 129.34, 127.86, 127.40, 124.06, 123.20, 122.92, 121.67, 120.90, 114.79, 111.27, 109.53, 102.15, 101.43, 98.84, 93.72, 55.82, 53.07, 51.67, 49.25, 37.91, 36.32, 33.01, 31.11, 29.62, 23.05, 21.02, 19.68, 17.76, 12.17, 11.35. LC-MS: 1145 [M+H] (FIG. 5).

### Synthesis of tert-butyl (6-aminohexyl)carbamate

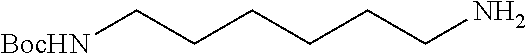 [SMILES: CC(C)(C)OC(=O)NCCCCCCN]

Di-tert-butyl dicarbonate (4.0 g, 18.4 mmol) was dissolved in chloroform and added drop-wise to a solution of hexamethylenediamine (10.6 g, 91.6 mmol) in chloroform at 0° C. The mixture was allowed to warm to room temperature. After stirring for 12 hours, the reaction crude was filtered and washed with chloroform. The filtrates were collected and solvent was evaporated. The residue was re-dissolved in ethyl acetate and washed with water and then brine. The organic solution was dried over anhydrous Na₂SO₄, filtered and concentrated under reduced pressure to afford 1.68 g, Yield: 42% of tert-butyl (6-aminohexyl)carbamate. ¹H NMR (CDCl₃, 400 MHz): δ 4.52 (bs, 1H), 3.10 (q, J=6.6 Hz, 2H), 2.68 (t, J=7.0 Hz, 2H), 1.49-1.30 (m, 17H), 1.25 (t, J=7.2 Hz, 2H). ¹³C NMR (100 MHz, CDCl₃): δ 156.1, 79.1, 42.2, 40.5, 33.8, 30.2, 28.4, 26.7, 26.6.

### Synthesis of Ce6-Hexane-NHBoc Conjugate (8)

Dimethyl ester of chlorin e6 4 (1 g, 1.60 mmol) was dissolved in anhydrous CH₂Cl₂ (30 mL). EDCl (614 mg, 3.2 mmol) and HOBt (432 mg, 3.2 mmol) were then added and allowed to stir until completely dissolved under nitrogen. After 30 min, tert-butyl (6-aminohexyl)carbamate (1.73 g, 8.0 mmol) and DIPEA (620 mg, 4.8 mmol) were mixed in CH₂Cl₂ (20 mL) and added to the reaction mixture. The mixture was allowed to stir at room temperature for 12 h under nitrogen. The reaction mixture was diluted with CH₂Cl₂ (200 mL) and then washed with brine and water, respectively. The organic layer was dried over anhydrous Na₂SO₄ and then evaporated. The product was purified via column chromatography to afford 700 mg of 8, Yield: 53%. UV-Vis (DMSO): λmax 670, 504, 408 nm. ¹H NMR (CDCl₃, 400 MHz): δ 9.63 (s, 1H), 9.58 (s, 1H), 8.74 (s, 1H), 8.03 (m, 1H), 6.31 (dd, J=16 Hz, 1H), 6.09 (dd, J=12 Hz, 1H), 5.49 (d, J=20 Hz, 1H), 5.21 (d, J=20 Hz, 1H), 4.40 and 4.26 (m, 2H), 3.85 (m, 5H), 3.58 (m, 8H), 3.42 (s, 3H), 3.25 (s, 3H), 3.10 (m, 2H), 1.69 and 2.12 (m, 2H), 1.74 (m, 2H), 1.67 (m, 6H), 1.50 (s, 17H), 1.36 (s, 2H), −1.71 (s, 1H), −1.92 (s, 1H). ¹³C NMR (CDCl₃, 100 MHz): δ 173.56, 169.39, 168.74, 166.67, 156.05, 154.15, 149.10, 144.74, 138.82, 136.12, 134.99, 134.75, 134.49, 130.14, 129.47, 128.43, 121.63, 102.15, 101.35, 98.85, 93.68, 79.08, 52.14, 51.65, 49.25, 40.49, 37.81, 31.14, 30.95, 29.48, 28.45, 26.76, 23.07, 19.70, 17.78, 14.22, 12.20, 11.37. LC-MS: 823 [M+H] (FIG. 6).

### Synthesis of Ce6-Hexane Amine Conjugate (9)

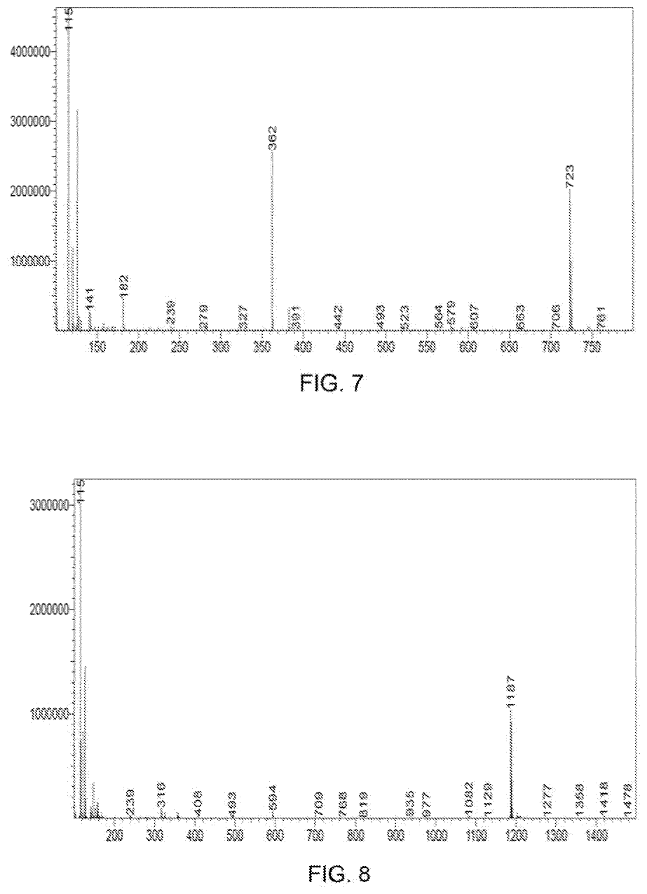

The compound 8 (700 mg, 0.85 mmol) was dissolved in of dry CH₂Cl₂ (30 mL) in an ice bath under argon. TFA (3 mL) was added, and the reaction mixture was stirred overnight. The reaction mixture was evaporated several times with diethyl ether to remove residual TFA. Then the precipitate was dissolved in CH₂Cl₂ and washed three times with H₂O and once with 10% NaHCO₃ to remove TFA. The organic layer was dried over anhydrous Na₂SO₄ and then evaporated to give a crude compound, purified by silica gel chromatography to give 350 mg of 9, Yield: 73%. UV-Vis (DMSO): λmax 658, 501, 400 nm. ¹H NMR (CDCl₃, 400 MHz): δ 9.63 (s, 1H), 9.58 (s, 1H), 8.73 (s, 1H), 8.01 (m, 1H), 6.31 (dd, J=16, 4 Hz, 1H), 6.09 (dd, J=12 Hz, 1H), 5.50 (d, J=20 Hz, 1H), 5.21 (d, J=20 Hz, 1H), 4.40 and 4.27 (m, 2H), 3.75 (m, 5H), 3.53 (s, 3H), 3.49 (s, 4H), 3.42 (s, 3H), 3.25 (s, 3H), 2.62 (t, J=4 & 8 Hz, 2H), 2.46 (m, 2H), 1.69 and 2.12 (m, 2H), 1.76-1.63 (m, 10H), 1.43 (m, 8H), −1.71 (s, 1H), −1.92 (s, 1H). ¹³C NMR (CDCl₃, 100 MHz): δ 173.57, 169.40, 168.75, 166.68, 154.15, 149.10, 144.74, 138.83, 136.14, 134.98, 134.80, 134.75, 134.49, 130.14, 129.45, 128.47, 121.63, 102.15, 101.35, 98.86, 93.69, 52.14, 51.65, 49.25, 41.97, 40.52, 37.78, 31.14, 29.45, 26.92, 23.08, 19.71, 17.78, 12.20, 11.37. LC-MS: 723 [M+H] (FIG. 7).

### Synthesis of Chlorine e6-curcumin Conjugate (10)

Compound 2 (300 mg, 0.62 mmol) was dissolved in dry CH₂Cl₂. A mixture of HOBt (100 mg, 0.74 mmol), EDCl (143 mg, 0.74 mmol), and DIPEA (66 mg, 0.51 mmol) in CH₂Cl₂ was added, and the mixture was allowed to stir for 30 min. Compound 9 (352 mg, 0.51 mmol) and DIPEA (160 mg, 1.24 mmol) were mixed in CH₂Cl₂ and added to this reaction mixture. The mixture was stirred overnight. It was diluted with CH₂Cl₂ and then washed with 5% aqueous citric acid, followed by a wash with brine and water. It was dried over anhydrous Na₂SO₄ and then evaporated. The residue was purified by silica gel column chromatography to afford 450 mg of 10, Yield: 61%. UV-Vis (DMSO): λmax 668, 505, 406 nm. ¹H NMR (CDCl₃, 400 MHz): δ 9.60 (s, 1H), 9.55 (s, 1H), 8.72 (s, 1H), 8.02 (m, 1H), 7.38 (m, 2H), 6.94-6.81 (m, 8H), 6.30-6.19 (m, 3H), 6.07 (dd, J=4 Hz, 1H), 5.50-5.44 (m, 2H), 5.23-5.17 (m, 2H), 4.40 and 4.26 (m, 2H), 3.86-3.78 (m, 5H), 3.73-3.71 (m, 9H), 3.54 (s, 4H), 3.46 (s, 4H), 3.41 (s, 3H), 3.27-3.24 (m, 6H), 2.54 (t, J=8 Hz, 2H), 2.50 (m, 1H), 2.26 (t, J=8 Hz, 2H), 2.10-1.99 (m, 5H), 1.74-1.62 (m, 9H), −1.71 (s, 1H), −1.92 (s, 1H); ¹³C NMR (CDCl₃, 100 MHz): δ 184.38, 181.46, 173.61, 172.21, 171.20, 169.51, 168.75, 166.68, 154.15, 151.08, 149.08, 147.91, 146.71, 144.75, 140.89, 138.99, 136.15, 134.95, 134.75, 134.72, 134.45, 133.96, 130.19, 129.51, 129.42, 128.33, 127.28, 124.11, 123.13, 122.84, 121.60, 120.90, 114.75, 111.29, 109.45, 102.10, 101.41, 98.84, 93.70, 55.84, 53.11, 52.18, 51.67, 49.23, 40.31, 39.18, 38.90, 37.78, 35.18, 32.83, 29.68, 26.52, 23.09, 21.14, 19.69, 17.78, 12.19, 11.36. LC-MS: 1187 [M+H] (FIG. 8).

### Synthesis of tert-butyl(2-(2-(2-aminoethoxy)ethoxy)ethyl)carbamate

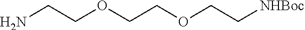 [SMILES: CC(C)(C)OC(=O)NCCOCCOCCN]

Under a nitrogen atmosphere, to a solution of 2,2′-(ethylenedioxy)-bis-(ethylamine) (14.8 g, 100 mmol) in anhydrous CHCl₃ (100 mL) cooled to 0° C. was added dropwise di-tert-butyldicarbonate (2.18 g, 10 mmol) in CHCl₃ (50 mL). After been stirred 24 h at room temperature, the solvent is evaporated under vacuum. The thick oil obtained is taken up in CH₂Cl₂ (100 mL). The organic layer is successively washed with saturated aqueous NaCl (50 mL), water (50 mL), dried over anhydrous Na₂SO₄ and concentrated in vacuo to afford 2.20 g, Yield: 89% of crude tert-butyl(2-(2-(2-aminoethoxy)ethoxy)ethyl)carbamate. This material was used without further purification. ¹H NMR (CDCl₃, 400 MHz): δ 5.15 (br s, 1H, NH), 3.63-3.51 (m, 8H), 3.31 (td, J=5 & 5 Hz, 2H), 2.88 (t, J=4.8 Hz, 2H), 1.45 (s, 9H), 1.40 (s, 2H, NH2); ¹³C NMR (CDCl₃, 100 MHz): d 155.42, 78.13, 72.80, 69.63, 41.08, 39.67, 27.77.

### Synthesis of Ce6-MonoPEG-NHBoc Conjugate (11)

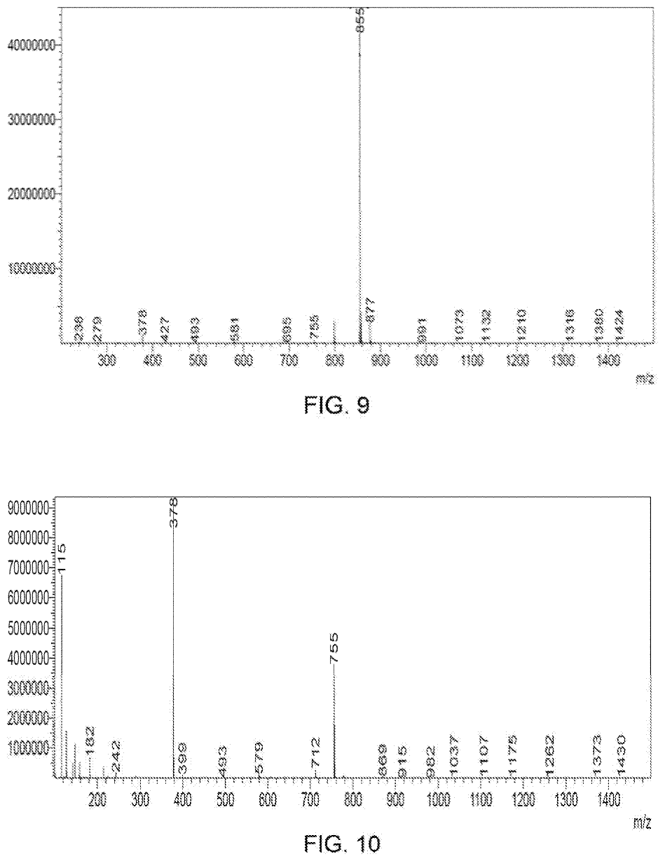

Dimethyl ester of chlorin e6 4 (1.5 g, 2.40 mmol) was dissolved in anhydrous CH₂Cl₂ (50 mL). EDCl (552 mg, 2.88 mmol) and HOBt (388 mg, 2.88 mmol) were then added and allowed to stir until completely dissolved under nitrogen. After 30 min, tert-butyl(2-(2-(2-aminoethoxy)ethoxy)ethyl)carbamate (2 g, 8.41 mmol) and DIPEA (620 mg, 4.8 mmol) were mixed in CH₂Cl₂ (20 mL) and added to the reaction mixture. The mixture was allowed to stir at room temperature for 12 h under nitrogen. The reaction mixture was diluted with CH₂Cl₂ (200 mL) and then washed with brine and water, respectively. The organic layer was dried over anhydrous Na₂SO₄ and then evaporated. The product was purified via column chromatography to afford 1.3 g of 11, Yield: 63%. UV-Vis (DMSO): λmax 667, 506, 410 nm. ¹H NMR (CDCl₃, 400 MHz): δ 9.63 (s, 1H), 9.57 (s, 1H), 8.73 (s, 1H), 8.02 (m, 1H), 6.31 (dd, J=16 Hz, 1H), 6.09 (dd, J=16 Hz, 1H), 5.51 (d, J=20 Hz, 1H), 5.24 (d, J=20 Hz, 1H), 4.66 and 4.02 (m, 2H), 4.42 and 4.31 (m, 2H), 3.84 (m, 2H), 3.74-3.66 (m, 7H), 3.54-3.50 (m, 9H), 3.29-3.25 (m, 5H), 2.97 (m, 2H), 2.50 and 2.17 (m, 2H), 1.74 and 2.08 (m, 2H), 1.67 (m, 8H), 1.50 (s, 9H) −1.71 (s, 1H), −1.92 (s, 1H). ¹³C NMR (CDCl₃, 100 MHz): δ 173.56, 169.51, 168.83, 166.69, 155.86, 154.24, 149.10, 144.75, 138.88, 136.09, 135.02, 134.78, 134.52, 130.16, 129.90, 128.26, 121.60, 102.32, 101.37, 98.83, 93.66, 79.12, 70.44, 69.86, 53.11, 52.19, 51.62, 49.22, 40.41, 37.89, 31.12, 29.70, 28.43, 23.09, 19.71, 17.76, 12.18, 11.36. LC-MS: 855 [M+H] (FIG. 9).

### Synthesis of Ce6-MonoPEGamine Conjugate (12)

The compound 11 (1.3 g, 1.52 mmol) was dissolved in of dry CH₂Cl₂ (30 mL) in an ice bath under argon. TFA (3 mL) was added, and the reaction mixture was stirred overnight. The reaction mixture was evaporated several times with diethyl ether to remove residual TFA. Then the precipitate was dissolved in CH₂Cl₂ and washed three times with H₂O and once with 10% NaHCO₃ to remove TFA. The organic layer was dried over anhydrous Na₂SO₄ and then evaporated to give a crude compound, purified by silica gel chromatography to give 1 g of 12, Yield: 87%. UV-Vis (DMSO): λmax 658, 501, 399 nm. ¹H NMR (CDCl₃, 400 MHz): δ 9.63 (s, 1H), 9.58 (s, 1H), 8.74 (s, 1H), 8.03 (m, 1H), 6.31 (dd, J=16, 4 Hz, 1H), 6.09 (dd, J=12 Hz, 1H), 5.54 (d, J=20 Hz, 1H), 5.25 (d, J=20 Hz, 1H), 4.42 and 4.00 (m, 2H), 4.28 and 4.01 (m, 2H), 3.86 (m, 2H), 3.75 (m, 6H), 3.66 (m, 2H) 3.54-3.49 (m, 9H), 3.43 (m, 2H), 3.26 (s, 3H), 3.21 (t, J=8 & 4 Hz, 2H), 2.49 and 2.10 (m, 2H), 1.69 and 2.10 (m, 2H), 1.67-1.63 (m, 6H), −1.71 (s, 1H), −1.92 (s, 1H); ¹³C NMR (CDCl₃, 100 MHz): δ 173.58, 169.57, 168.79, 166.78, 154.08, 149.06, 144.71, 138.80, 136.14, 135.08, 134.83, 134.73, 134.49, 130.12, 129.51, 128.60, 121.63, 102.36, 101.26, 98.84, 93.68, 72.78, 70.47, 69.98, 53.10, 52.19, 51.66, 49.22, 41.05, 40.32, 37.75, 31.11, 29.68, 23.12, 19.73, 17.78, 12.21, 11.39. LC-MS: 755 [M+H] (FIG. 10).

### Synthesis of Chlorine e6-curcumin Conjugate (13)

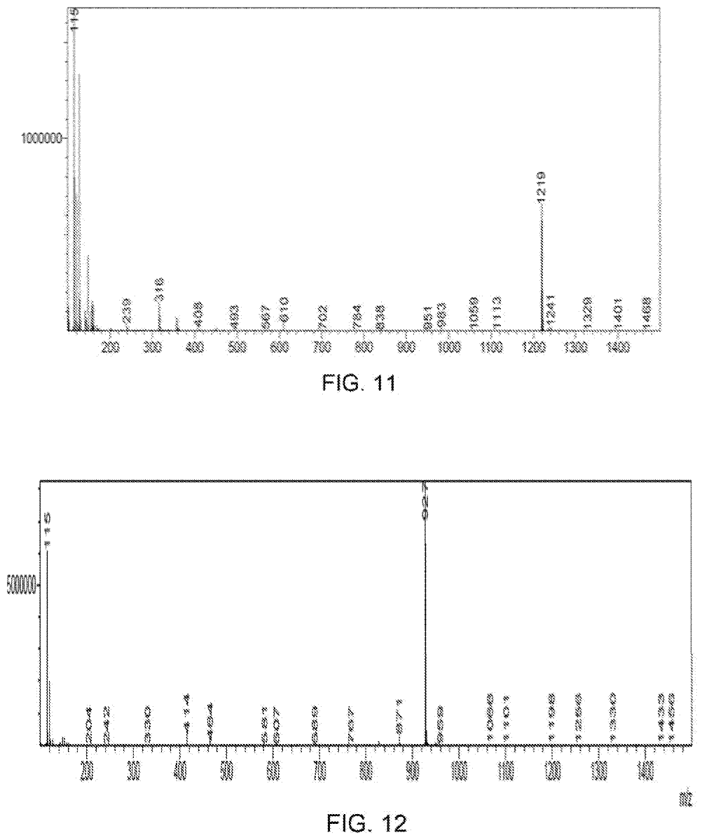

Compound 2 (700 mg, 1.45 mmol) was dissolved in dry CH₂Cl₂. A mixture of HOBt (235 mg, 1.74 mmol), EDCl (333 mg, 1.74 mmol), and DIPEA (187 mg, 1.45 mmol) in CH₂Cl₂ was added, and the mixture was allowed to stir for 30 min. Compound 12 (1.09 g, 1.45 mmol) and DIPEA (187 mg, 1.45 mmol) were mixed in CH₂Cl₂ and added to this reaction mixture. The mixture was stirred overnight. It was diluted with CH₂Cl₂ and then washed with 5% aqueous citric acid, followed by a wash with brine and water. It was dried over anhydrous Na₂SO₄ and then evaporated. The residue was purified by silica gel column chromatography to afford 770 mg of 13, Yield: 44%. UV-Vis (DMSO): λmax 668, 502, 406 nm. ¹H NMR (CDCl₃, 400 MHz): δ 9.62 (s, 1H), 9.56 (s, 1H), 8.73 (s, 1H), 8.01 (m, 1H), 7.48 (d, J=16H, 1H), 7.37 (d, J=16H, 1H), 7.01-6.99 (m, 2H), 6.93-6.83 (m, 4H), 6.71 (d, J=8 Hz, 1H), 6.31-6.24 (m, 2H), 6.08 (d, J=12 Hz, 1H), 5.56-5.48 (m, 2H), 5.25-5.12 (m, 2H), 4.40 and 4.26 (m, 2H), 4.01 (m, 1H), 3.83 (m, 6H), 3.73 (m, 6H), 3.64 (m, 2H), 3.59 (s, 3H), 3.49-3.47 (m, 6H), 3.41 (s, 3H), 3.25 (s, 3H), 3.07 (m, 2H), 2.70 (m, 2H), 2.50 (m, 2H), 2.14-2.06 (m, 6H), 1.94 (t, J=4 & 8 Hz, 2H), 1.71-1.58 (m, 8H), −1.71 (s, 1H), −1.92 (s, 1H). ¹³C NMR (CDCl₃, 100 MHz): δ 184.36, 181.56, 173.58, 172.01, 170.82, 169.56, 169.00, 166.67, 151.03, 147.96, 146.76, 144.78, 140.90, 140.87, 139.08, 136.21, 134.96, 134.84, 134.60, 133.79, 130.35, 129.79, 129.37, 128.27, 127.31, 124.01, 123.00, 122.87, 121.74, 120.76, 114.83, 111.16, 109.54, 102.28, 101.33, 98.84, 93.81, 70.29, 69.55, 55.76, 53.10, 52.26, 51.68, 49.22, 40.39, 38.59, 37.79, 34.26, 31.12, 29.69, 23.12, 20.47, 19.69, 17.76, 12.17, 11.36. LC-MS: 1219 [M+H] (FIG. 11).

### Synthesis of tert-butyl (3-(2-(2-(3-aminopropoxy)ethoxy)ethoxy) propyl)carbamate

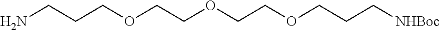 [SMILES: CC(C)(C)OC(=O)NCCCOCCOCCOCCCN]

A solution of 4,7,10-trioxa-1,13-tridecanediamine (7.5 g, 34.1 mmol) in 1,4-dioxane (100 mL) was treated with BOC-anhydride (3.7 g, 16.9 mL). The mixture was stirred at room temperature for 12 h. The solvent was removed, and the resulting yellow oil was purified by silica gel flash chromatography to produce the oil 5.5 g of tert-butyl (3-(2-(2-(3-aminopropoxy)ethoxy)ethoxy)propyl)carbamate, Yield: 49%. ¹H NMR (CDCl₃, 400 MHz): δ 5.1 (s, 1H), 3.58-3.50 (m, 12H), 3.21 (d, J=6.9 Hz, 2H), 2.79 (t, J=8 Hz, 2H), 1.75-1.69 (m, 4H), 1.59 (s, 2H), 1.42 (s, 9H). ¹³C NMR (CDCl₃, 100 MHz): δ 155.0, 69.2, 68.9, 66.7, 48.0, 37.6, 30.4, 28.6, 27.3.

### Synthesis of Ce6-diPEG-NHBoc Conjugate (14)

Dimethyl ester of chlorin e6 2 (1 g, 1.60 mmol) was dissolved in anhydrous CH₂Cl₂ (50 mL). EDCl (368 mg, 1.92 mmol) and HOBt (260 mg, 1.92 mmol) were then added and allowed to stir until completely dissolved under nitrogen. After 30 min, tert-butyl (3-(2-(2-(3-aminopropoxy)ethoxy)ethoxy)propyl)carbamate (1.28 g, 4 mmol) and DIPEA (414 mg, 3.20 mmol) were mixed in CH₂Cl₂ (20 mL) and added to the reaction mixture. The mixture was allowed to stir at room temperature for 12 h under nitrogen. The reaction mixture was diluted with CH₂Cl₂ (200 mL) and then washed with brine and water, respectively. The organic layer was dried over anhydrous Na₂SO₄ and then evaporated. The product was purified via column chromatography to afford 1.2 g of 14, Yield: 81%. UV-Vis (DMSO): λmax 667, 506, 410 nm. ¹H NMR (CDCl₃, 400 MHz): δ 9.68 (s, 1H), 9.63 (s, 1H), 8.79 (s, 1H), 8.07 (m, 1H), 6.37 (dd, J=20 Hz, 1H), 6.14 (dd, J=14.8 Hz, 1H), 5.61 (d, J=20 Hz, 1H), 5.27 (d, J=16 Hz, 1H), 4.44-4.34 (m, 3H), 4.05 (m, 1H), 3.82-3.72 (m, 8H), 3.63-3.48 (m, 12H), 3.31 (s, 5H), 2.70 (s, 2H), 2.60-2.49 (m, 3H), 2.35 (m, 2H), 2.20-2.09 (m, 6H), 1.78-1.70 (m, 8H), 1.37 (s, 9H) −1.64 (s, 1H), −1.94 (s, 1H); ¹³C NMR (CDCl₃, 100 MHz): δ 173.55, 169.44, 168.86, 166.76, 154.15, 148.99, 144.79, 138.82, 136.18, 135.01, 134.92, 134.74, 134.62, 130.25, 129.75, 129.35, 128.31, 121.84, 102.15, 101.27, 98.87, 93.69, 79.08, 70.12, 69.67, 69.05, 68.28, 53.07, 52.45, 51.68, 49.07, 39.09, 37.60, 31.06, 29.75, 29.00, 23.14, 19.56, 17.73, 12.18, 11.29. LC-MS: 927 [M+H] (FIG. 12).

### Synthesis of Ce6-diPEGamine Conjugate (15)

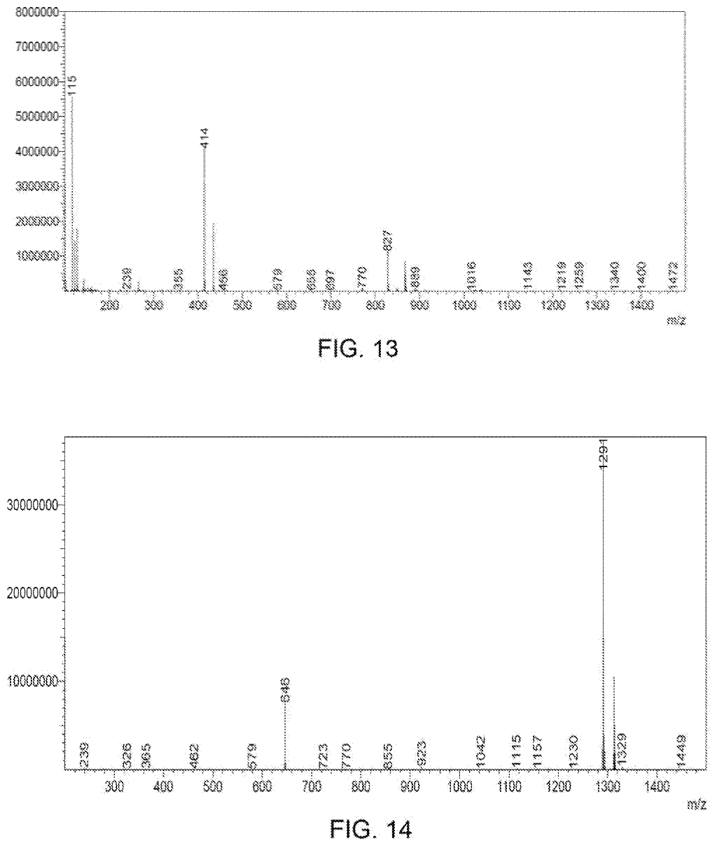

The compound 14 (1.2 g, 1.52 mmol) was dissolved in of dry CH₂Cl₂ (30 mL) in an ice bath under argon. TFA (5 mL) was added, and the reaction mixture was stirred overnight. The reaction mixture was evaporated several times with diethyl ether to remove residual TFA. Then the precipitate was dissolved in CH₂Cl₂ and washed three times with H₂O and once with 10% NaHCO₃ to remove TFA. The organic layer was dried over anhydrous Na₂SO₄ and then evaporated to give a crude compound, purified by silica gel chromatography to give 1 g of 15, Yield: 93%. UV-Vis (DMSO): λmax 657, 501, 400 nm. ¹H NMR (CDCl₃, 400 MHz): δ 9.63 (s, 1H), 9.58 (s, 1H), 8.78 (s, 1H), 8.04 (m, 1H), 6.33 (dd, J=16 Hz, 1H), 6.12 (dd, J=12 Hz, 1H), 5.58 (d, J=20 Hz, 1H), 5.31 (d, J=16 Hz, 1H), 4.43 and 4.29 (m, 2H), 3.82-3.76 (m, 5H), 3.63-3.56 (m, 9H), 3.45-3.39 (m, 9H) 3.26-3.22 (m, 5H), 2.76 (m, 2H), 2.18-2.13 (m, 8H), 1.73-1.61 (m, 8H), −1.76 (s, 1H), −1.96 (s, 1H); ¹³C NMR (CDCl₃, 100 MHz): δ 173.55, 169.44, 168.86, 166.75, 154.15, 148.99, 144.79, 138.82, 136.18, 135.01, 134.92, 134.74, 134.62, 130.25, 129.75, 129.35, 128.31, 121.84, 102.15, 101.27, 98.87, 93.69, 69.98, 69.67, 68.28, 53.07, 52.45, 51.68, 49.07, 39.09, 37.60, 31.06, 29.75, 23.14, 19.56, 17.73, 12.18, 11.29. LC-MS: 827 [M+H] (FIG. 13).

### Synthesis of Chlorine e6-curcumin Conjugate (16)

Compound 2 (500 mg, 1.45 mmol) was dissolved in dry CH₂Cl₂. A mixture of HOBt (167 mg, 1.23 mmol), EDCl (236 mg, 1.23 mmol), and DIPEA (133 mg, 1.03 mmol) in CH₂Cl₂ was added, and the mixture was allowed to stir for 30 min. Compound 15 (856 mg, 1.03 mmol) and DIPEA (133 mg, 1.03 mmol) were mixed in CH₂Cl₂ and added to this reaction mixture. The mixture was stirred overnight. It was diluted with CH₂Cl₂ and then washed with 5% aqueous citric acid, followed by a wash with brine and water. It was dried over anhydrous Na₂SO₄ and then evaporated. The residue was purified by silica gel column chromatography to afford 770 mg of 16, Yield: 41%. UV-Vis (DMSO): λmax 668, 502, 407 nm. ¹H NMR (400 MHz, CDCl₃): δ 9.67 (s, 1H), 9.63 (s, 1H), 8.80 (s, 1H), 8.09 (m, 1H), 7.53 (d, J=16H, 1H), 7.46 (d, J=16H, 1H), 7.20 (m, 1H), 7.06-7.04 (m, 2H), 6.96-6.84 (m, 4H), 6.37-6.32 (m, 3H), 6.15 (dd, J=12 Hz, 1H), 5.65-5.57 (m, 2H), 5.34-5.23 (m, 2H), 4.49 and 4.37 (m, 2H), 4.01 (m, 1H), 3.92-3.89 (m, 4H), 3.82-3.69 (m, 12H), 3.62 (s, 3H), 3.55-3.51 (m, 6H), 3.47 (s, 3H), 3.38-3.36 (m, 2H), 3.31 (s, 3H), 2.89 (m, 2H), 2.64-2.52 (m, 3H), 2.43-2.33 (m, 6H), 2.25-2.09 (m, 5H), 1.84-1.69 (m, 9H), −1.66 (s, 1H), −1.89 (s, 1H). ¹³C NMR (CDCl₃, 100 MHz): δ 184.35, 181.66, 173.54, 171.56, 170.96, 169.28, 168.76, 166.90, 154.04, 151.13, 149.07, 147.97, 146.77, 144.74, 140.99, 139.15, 138.75, 136.18, 135.09, 134.80, 134.67, 134.48, 133.87, 130.19, 129.92, 129.43, 128.74, 127.42, 124.09, 123.09, 122.96, 121.71, 121.58, 120.81, 114.81, 111.26, 109.55, 102.42, 101.44, 101.23, 98.80, 93.72, 70.30, 69.99, 69.12, 55.86, 53.10, 52.14, 51.67, 49.19, 39.11, 37.72, 34.84, 32.91, 31.16, 29.67, 29.27, 28.15, 23.09, 20.77, 19.71, 17.81, 12.20, 11.38. LC-MS: 1291 [M+H] (FIG. 14).

What is claimed is:

1. A chlorin e6-curcumin derivative selected from the group consisting of Formula I, II, III and IV:

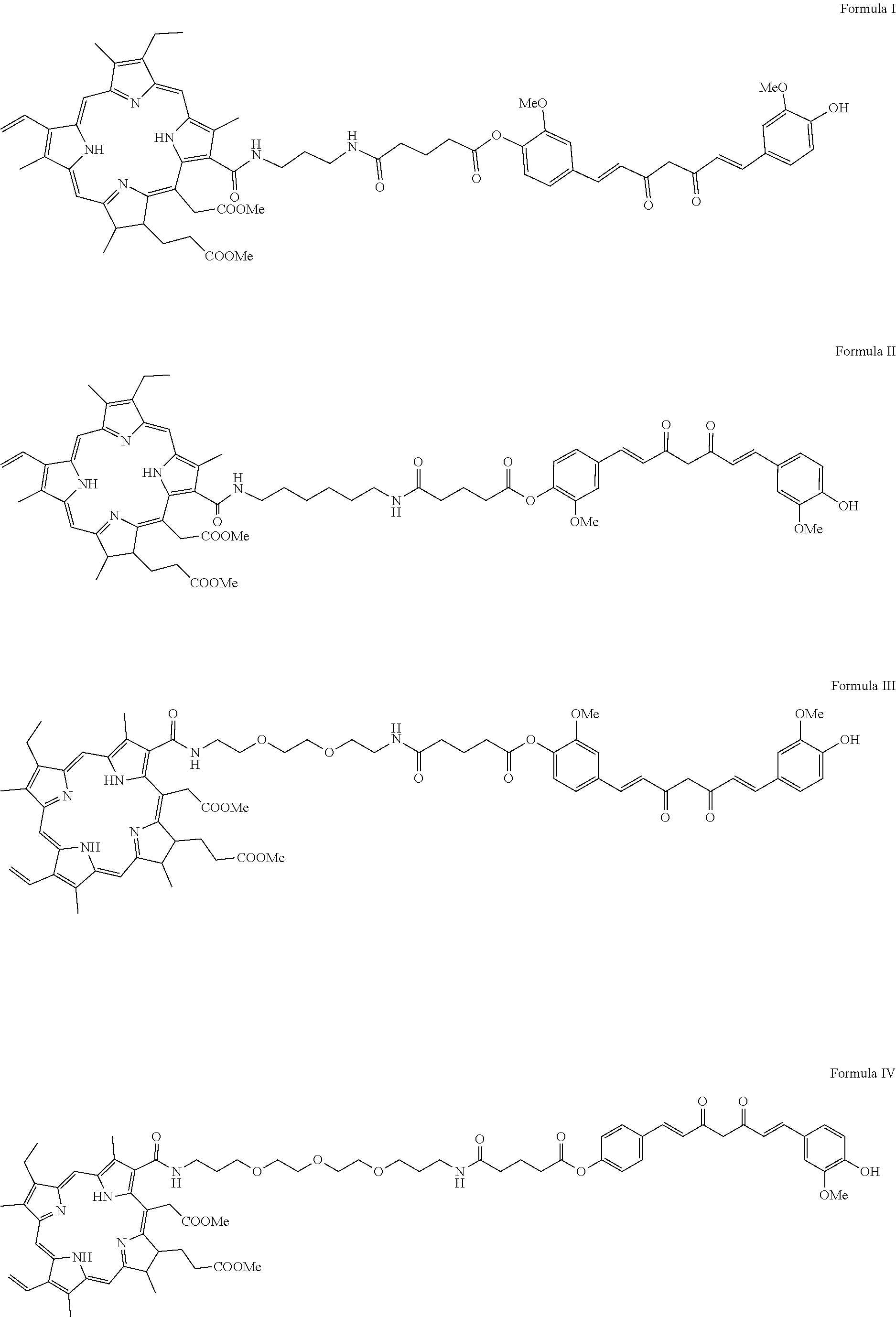

2. A method for preparing a chlorine e6-curcumin derivative selected from the group consisting of Formula I, II, III and IV and preparation of its intermediates compounds 5, 6, 8, 9, 11, 12, 14, and 15.

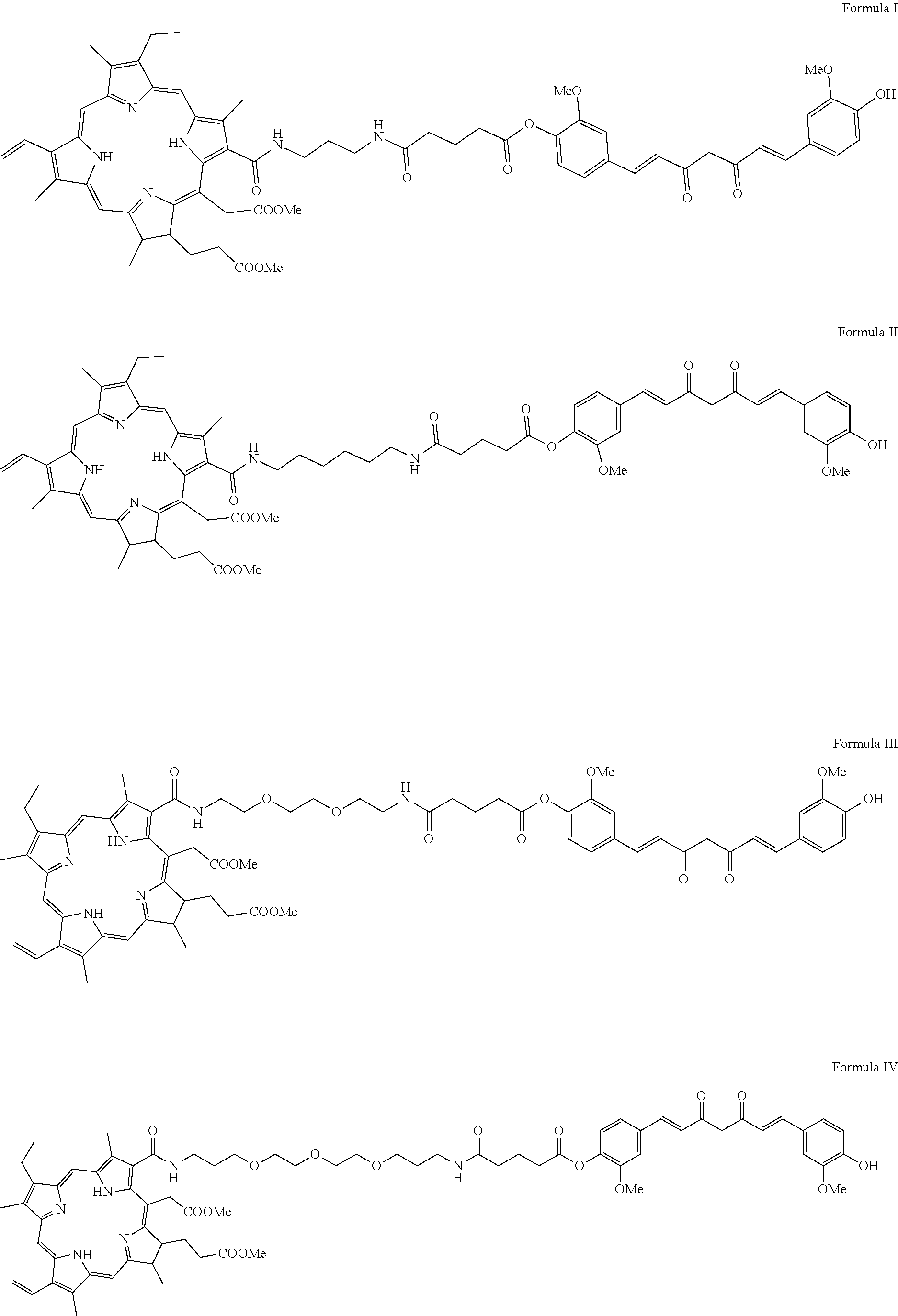

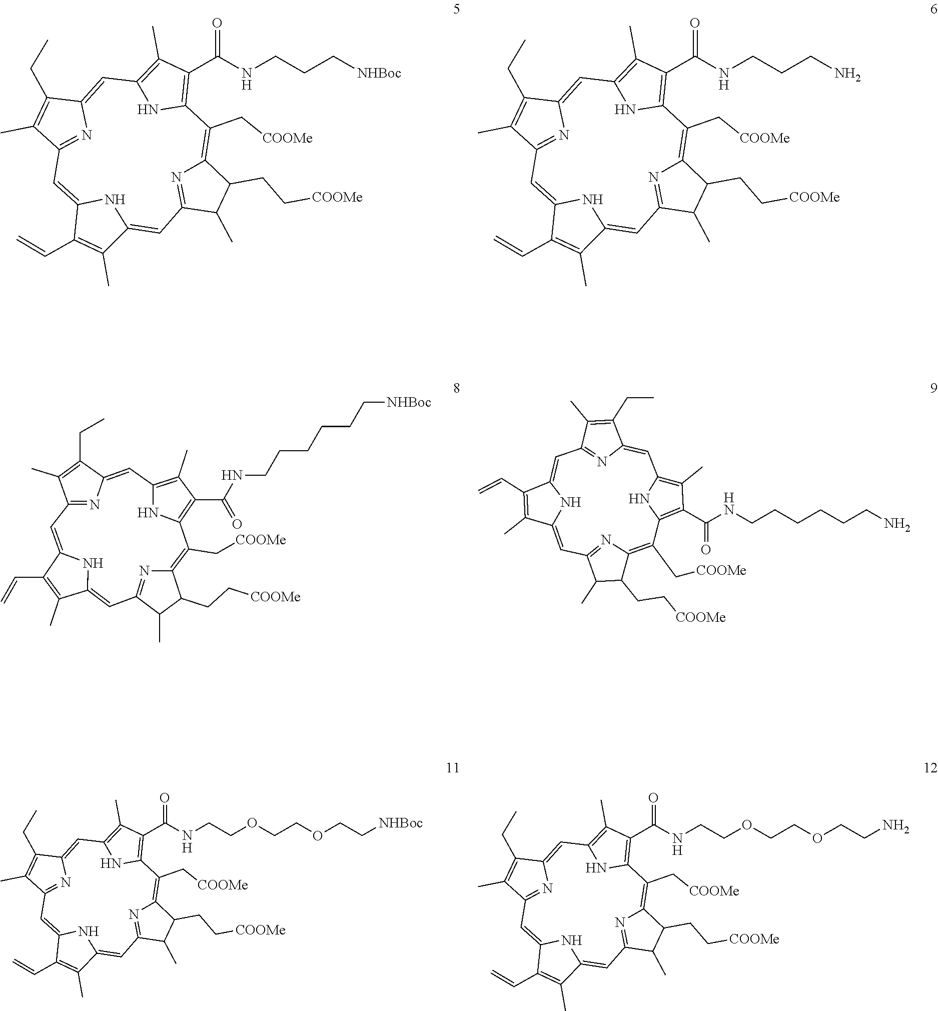

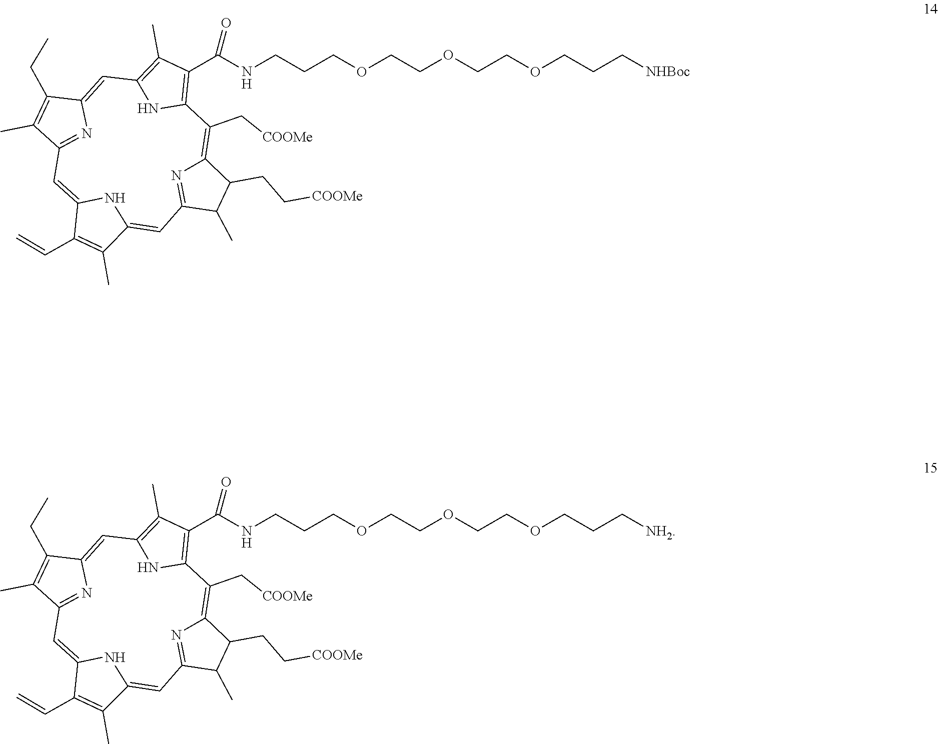 [SMILES: C=Cc1c(C)c2cc3nc(c(CC(=O)OC)c4[nH]c(cc5nc(cc1[nH]2)C(C)=C5CC)c(C)c4C(=O)NCCCOCCOCCOCCCNC(=O)OC(C)(C)C)C(CCC(=O)OC)C3C ; C=Cc1c(C)c2cc3nc(c(CC(=O)OC)c4[nH]c(cc5nc(cc1[nH]2)C(C)=C5CC)c(C)c4C(=O)NCCCOCCOCCOCCCN)C(CCC(=O)OC)C3C]

3. A method for treating cancer, comprising:
administering to a subject in need thereof a therapeutically effective amount of a compound selected from the group consisting of Formula I, II, III, and IV: [chemical structure]
